(19) United States
(12) Patent Application Publication
(10) Pub. No.: US 2025/0340950 A1
(43) Pub. Date: Nov. 6, 2025

(54) JOINT MODELING OF LONGITUDINAL AND TIME-TO-EVENT DATA TO PREDICT PATIENT SURVIVAL

(71) Applicant: GUARDANT HEALTH, INC., Palo Alto, CA (US)

(72) Inventors: Christopher PRETZ, Denver, CO (US); Aaron Isaac HARDIN, San Francisco, CA (US); Amar DAS, Pasadena, CA (US)

(21) Appl. No.: 19/265,371

(22) Filed: Jul. 10, 2025

Related U.S. Application Data
(63) Continuation of application No. PCT/US2024/011186, filed on Jan. 11, 2024.
(60) Provisional application No. 63/479,470, filed on Jan. 11, 2023.
(60) Provisional application No. 63/496,765, filed on Apr. 18, 2023.
(60) Provisional application No. 63/612,218, filed on Dec. 19, 2023.

(51) Int. Cl. C12Q 1/6886; C12Q 1/6806; G16H 10/60

(52) U.S. Cl. CPC C12Q 1/6886; C12Q 1/6806; G16H 10/60; C12Q 2600/156

(57) ABSTRACT

Changes in ctDNA levels can fluctuate significantly over time from patient to patient, and the results can be difficult to interpret. Described herein are methods and techniques capable of capturing these complexities while accounting for a diverse set of patient traits. Furthermore, analyzing an observational dataset consisting of patients with cancer who received therapy, analytic results are capable of being presented graphically. These results demonstrate the utility of the described methods and techniques in acquiring a comprehensive understanding of how response patterns evolve and how different patient characteristics influence these evolutions.

### CROSS-REFERENCE TO RELATED APPLICATION

The present application is a Continuation of International Patent Application No. PCT/US2024/011186, filed Jan. 11, 2024, which claims the benefit of priority to U.S. Provisional Application Ser. No. 63/479,470, filed on Jan. 11, 2023, U.S. Provisional Application Ser. No. 63/496,765, filed on Apr. 18, 2023, U.S. Provisional Application Ser. No. 63/612,218, filed on Dec. 19, 2023, which are each incorporated by reference herein in their entirety.

### BACKGROUND

Today, there is increasing knowledge of the molecular pathogenesis of cancer and with next generation sequencing techniques, increases in the potential to study early molecular alterations in cancer development. This includes liquid biopsy in body fluids. Genetic and epigenetic alterations associated with cancer development can be found in cell-free DNA (cfDNA), such as those in plasma, serum, urine, etc. with the potential for use as diagnostic biomarkers. Non-invasive sampling methods foster patient compliance, as easier, faster, and more economical to perform.

Such liquid biopsy techniques support characterization of the genomic makeup of different tissues in the subject. While generally released by all types of cells, cfDNA can originate from necrotic or apoptotic cells, for identification of specific tumor-related alterations, such as mutations, methylation, and copy number variations (CNVs). Improved characterization of this circulating tumor DNA (ctDNA) is challenging given the need to differentiate the signal originating from a disease tissue, such as cancer, from signals originating from germline cells releasing cfDNA the wider range of tissues, such as healthy tissue and white blood cells undergoing hematopoiesis. One can enrich signals by identifying variant alleles having allele fractions that do not adhere to exemplary 1:1 ratios for heterozygous alleles in the germline.

Despite these advances, the majority of cfDNA use as diagnostics focus on advanced tumor stages, with much less known as to characteristics of early malignant disease stages. Yet several hurdles exist for early stage detection, including smaller numbers of aberrations, confounding phenomena such as clonal non-tumorous tissue expansion, adventitious cancer-associated mutations, and lack of understanding as to significance of driver alterations. Thus, there is a great need in the art for improved techniques for characterizing early disease stages to support development of cfDNA and ctDNA related diagnostics.

Described herein is use of detection measurement, which can include a variety of parameters including longitudinal and time-to-event data, thereby supporting understanding of how temporal changes in a biomarker relate to a time-to-event response and patient outcomes. For example, methods and techniques described herein incorporate longitudinal and time-to-event data supports to decipher temporal changes in a biomarker as related to a time-to-event response. Additionally, methods and techniques described herein allows evaluation of patient characteristics such as age, gender, etc. in analyses. Repeated measures via liquid biopsy provide an opportunity to assess patient outcomes.

### BRIEF DESCRIPTION OF THE FIGURES

FIG. 1. Distribution of Allele Frequency and Tumor Fraction. FIG. 1A. Depiction of allele frequency and tumor fraction for EGFR L858R. FIG. 1B. Depiction of allele frequency and log transformation for EGFR L858R, KRAS G12D, KRAS G12V.

FIG. 2. Spaghetti Plot of Allele Frequency and Tumor Percent. FIG. 2A. Spaghetti plot of allele frequency and tumor percent for EGFR L858R. FIG. 2B. Spaghetti plot of allele frequency and log transformation for EGFR L858R, KRAS G12D, KRAS G12V.

FIG. 3. Fitted Cubic Spline Based GLMM Results for Log Transformed Biomarkers for EGFR L858R.

FIG. 4. The Biomarker Evolution and Corresponding Survival Curves. Biomarker EGFR L858R evolution shown for a patient at 300 days, 600 days, and 900 days.

FIG. 5. The Biomarker Evolution and Corresponding Survival Curves. Biomarker EGFR L858R evolution shown for a patient at 300 days, 600 days, and 900 days.

FIG. 6. The Biomarker Evolution and Corresponding Survival Curves. Biomarker KRAS G12V evolution shown for a patient at 300 days, 600 days, and 900 days.

FIG. 7. Time-to-Event Sub-Models: Overall Survival. Depiction for EGFR L858R, KRAS G12D, KRAS G12V.

FIG. 8. Random Effects Modeling. Depiction for EGFR L858R, KRAS G12D, KRAS G12V.

FIG. 9. Distribution of ctDNA levels and Logit Transformed ctDNA Levels and Corresponding Spaghetti Plots.

FIG. 10. Unconditional Model Fit with and without Datapoints for the non-small cell lung cancer (NSCLC) Cohort. Unconditional Model Fit with and without Datapoints for the NSCLC Cohort. The black curve denotes the response pattern for the cohort, while each black dot indicates a ctDNA level value. The purple region represents the 95% confidence bands of the estimated trajectory.

FIG. 11. Response Patterns for Different Values of Baseline Age and ELIX Scores for: FIG. 11A. Alive and FIG. 11B. Deceased Patients for Female Non-Smokers Receiving their First Line of anti-EGFR Treatment.

FIG. 12. Velocity (IRC) Plots for Different Values of Baseline Age and ELIX Scores for: FIG. 12A. Alive and FIG. 12B. Deceased Patients for Female Non-Smokers Receiving their First Line of anti-EGFR Treatment.

### SUMMARY OF THE INVENTION

Described herein is a method of determining a patient response in at least one patient, comprising, obtaining nucleic acid sequence information from at least one patient, comprising measurements of temporal changes in a biomarker; and determining a patient response for the at least one patient. In various embodiments, the biomarker comprises ctDNA. In various embodiments, the biomarker comprises allele frequency and tumor fraction. In various embodiments, the method includes determining a patient response for the at least one patient comprises use of a database. In various embodiments, the method includes database comprises medical records and/or insurance records. In various embodiments, the method includes use of the database comprises application of a model. In various embodiments, the model is a hierarchal model. In various embodiments, the model is an effects model. In various embodiments, the model is a regression model. In various embodiments, the model is a joint model. In various embodiments, the hierarchal model is a hierarchical random effects model. In various embodiments, the model comprises a cubic spline. In various embodiments, the model comprises a regression model. In various embodiments, the hierarchal random effects model comprises generation of data from nucleic acid sequence information comprising temporal changes in a biomarker comprising circulating tumor DNA (ctDNA) from at least one subject in a plurality of subjects. In various embodiments, the generation of data comprises generation of a cubic spline for at least one subject in a plurality of subjects. In various embodiments, the generation of data comprises generation of response parameters comprising one or more covariates. In various embodiments, the generation of data comprises generation of response parameters without covariates. In various embodiments, the response parameters apply a multivariate normal distribution. In various embodiments, the method includes determining a patient response for the at least one patient comprises generation of a velocity plot. In various embodiments, the method includes determining a patient response for the at least one patient comprises comparison to the model. In various embodiments, the joint model comprises at least two models. In various embodiments, the joint model comprises association factors between the at least two models. In various embodiments, the joint model comprises a cubic spline and a proportional hazard model. In various embodiments, the biomarker is measured with next-generation DNA sequencing. In various embodiments, next-generation DNA sequencing comprising ligation of non-unique barcodes to the ctDNA. In various embodiments, next-generation DNA sequencing comprising ligation of unique barcodes to the ctDNA. In various embodiments, next-generation DNA sequencing comprising ligation of non-unique barcodes to ctDNA fragments, wherein the non-unique barcodes are present in at least 20×, at least 30×, at least 50×, or at least 100× molar excess.

A system comprising a machine comprising at least one processor and storage comprising instructions capable of performing any of the preceding methods. A computer readable medium comprising instructions capable of performing any of the preceding methods.

Described herein is a method of determining a patient response in at least one patient, comprising, obtaining nucleic acid sequence information from at least one patient, comprising measurements of temporal changes in a biomarker comprising circulating tumor DNA (ctDNA); and determining a patient response for the at least one patient comprising use of a database comprising medical records and/or insurance record from a plurality of subjects wherein use of the database comprises application of a hierarchal random effects model. In various embodiments, the hierarchal random effects model comprises generation of data from nucleic acid sequence information comprising temporal changes in ctDNA from at least one subject in a plurality of subjects. In various embodiments, the hierarchal random effects model comprises generation of a cubic spline for at least one subject in the plurality of subjects. In various embodiments, the hierarchal random effects model comprises response parameters comprising one or more covariates for at least one subject in the plurality of subjects. In various embodiments, the database comprises medical records and/or insurance records for the plurality of subjects. Described herein is a system comprising a machine comprising at least one processor and storage comprising instructions capable of performing a method of determining a patient response in at least one patient, comprising, obtaining nucleic acid sequence information from at least one patient, comprising measurements of temporal changes in a biomarker comprising circulating tumor DNA (ctDNA); and determining a patient response for the at least one patient comprising use of a database comprising medical records and/or insurance record from a plurality of subjects wherein use of the database comprises application of a hierarchal random effects model. In various embodiments, the hierarchal random effects model comprises generation of data from nucleic acid sequence information comprising temporal changes in ctDNA from at least one subject in a plurality of subjects. In various embodiments, the hierarchal random effects model comprises generation of a cubic spline for at least one subject in the plurality of subjects. In various embodiments, the hierarchal random effects model comprises response parameters comprising one or more covariates for at least one subject in the plurality of subjects. In various embodiments, the database comprises medical records and/or insurance records for the plurality of subjects. Described herein is a computer readable medium comprising instructions capable of performing a method of determining a patient response in at least one patient, comprising, obtaining nucleic acid sequence information from at least one patient, comprising measurements of temporal changes in a biomarker comprising circulating tumor DNA (ctDNA); and determining a patient response for the at least one patient comprising use of a database comprising medical records and/or insurance record from a plurality of subjects wherein use of the database comprises application of a hierarchal random effects model. In various embodiments, the hierarchal random effects model comprises generation of data from nucleic acid sequence information comprising temporal changes in ctDNA from at least one subject in a plurality of subjects. In various embodiments, the hierarchal random effects model comprises generation of a cubic spline for at least one subject in the plurality of subjects. In various embodiments, the hierarchal random effects model comprises response parameters comprising one or more covariates for at least one subject in the plurality of subjects. In various embodiments, the database comprises medical records and/or insurance records for the plurality of subjects.

Described herein is a method of determining a patient response in at least one patient, comprising, obtaining nucleic acid sequence information from at least one patient, comprising measurements of temporal changes in a biomarker comprising circulating tumor DNA (ctDNA); and determining a patient response for the at least one patient comprising use of a database comprising medical records and/or insurance record from a plurality of subjects wherein use of the database comprises application of a joint model comprising a cubic spline and proportional hazard model generated from data from nucleic acid sequence information for at least one subject in a plurality of subjects. In various embodiments, the database comprises medical records and/or insurance records for the plurality of subjects. Described herein is a system comprising a machine comprising at least one processor and storage comprising instructions capable of performing is a method of determining a patient response in at least one patient, comprising, obtaining nucleic acid sequence information from at least one patient, comprising measurements of temporal changes in a biomarker comprising circulating tumor DNA (ctDNA); and determining a patient response for the at least one patient comprising use of a database comprising medical records and/or insurance record from a plurality of subjects wherein use of the database comprises application of a joint model comprising a cubic spline and proportional hazard model generated from data from nucleic acid sequence information for at least one subject in a plurality of subjects. In various embodiments, the database comprises medical records and/or insurance records for the plurality of subjects. Described herein is a computer readable medium comprising instructions capable of performing is a method of determining a patient response in at least one patient, comprising, obtaining nucleic acid sequence information from at least one patient, comprising measurements of temporal changes in a biomarker comprising circulating tumor DNA (ctDNA); and determining a patient response for the at least one patient comprising use of a database comprising medical records and/or insurance record from a plurality of subjects wherein use of the database comprises application of a joint model comprising a cubic spline and proportional hazard model generated from data from nucleic acid sequence information for at least one subject in a plurality of subjects. In various embodiments, the database comprises medical records and/or insurance records for the plurality of subjects.

### DETAILED DESCRIPTION

### Analysis

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic and other analyte data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

The present analyses are also useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence of certain diseases. Clonal expansions may be monitored using copy number variation detection and certain immune states may be monitored. In this example, copy number variation analysis may be performed over time to produce a profile of how a particular disease may be progressing. Copy number variation or even rare mutation detection may be used to determine how a population of pathogens changes during the course of infection. This may be particularly important during chronic infections, such as HIV/AIDS or Hepatitis infections, whereby viruses may change life cycle state and/or mutate into more virulent forms during the course of infection. The present methods may be used to determine or profile rejection activities of the host body, as immune cells attempt to destroy transplanted tissue to monitor the status of transplanted tissue as well as altering the course of treatment or prevention of rejection.

For example, numerous types of malfunctions and abnormalities that commonly occur in the cardiovascular system, wherein failure to diagnose or treat, will progressively decrease the body's ability to supply sufficient oxygen to satisfy the coronary oxygen demand when the individual encounters stress. The progressive decline in the cardiovascular system's ability to supply oxygen under stress conditions will ultimately culminate in a heart attack, i.e., myocardial infarction event that is caused by the interruption of blood flow through the heart resulting in oxygen starvation of the heart muscle tissue (i.e., myocardium). In many cases, permanent damage will occur to the cells comprising the myocardium that will subsequently predispose the individual's susceptibility to additional myocardial infarction events.

Methods of the disclosure can characterize malfunctions and abnormalities associated with the heart muscle and valve tissues (e.g., hypertrophy), the decreased supply of blood flow and oxygen supply to the heart are often secondary symptoms of debilitation and/or deterioration of the blood now and supply system caused by physical and biochemical stresses. Examples of cardiovascular diseases that are directly affected by these types of stresses include atherosclerosis, coronary artery disease, peripheral vascular disease and peripheral artery disease, along with various cardias and arrythmias which may represent other forms of disease and dysfunction.

Further, the methods of the disclosure may be used to characterize the heterogeneity of an abnormal condition in a subject. Such methods can include, e.g., generating a genetic profile of extracellular polynucleotides derived from the subject, wherein the genetic profile includes a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation and mutation analyses alone or in combination.

The present methods can be used to diagnose, prognose, monitor or observe cancers. or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

### Methods of Modified Nucleic Acid Analysis

The disclosure provides alternative methods for analyzing modified nucleic acids (e.g., methylated, linked to histones and other modifications discussed above). In some such methods, a population of nucleic acids bearing the modification to different extents (e.g., 0, 1, 2, 3, 4, 5 or more methyl groups per nucleic acid molecule) is contacted with adapters before fractionation of the population depending on the extent of the modification. Adapters attach to either one end or both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. Following attachment of adapters, the nucleic acids are amplified from primers binding to the primer binding sites within the adapters. Adapters, whether bearing the same or different tags, can include the same or different primer binding sites, but preferably adapters include the same primer binding site. Following amplification, the nucleic acids are contacted with an agent that preferably binds to nucleic acids bearing the modification (such as the previously described such agents). The nucleic acids are separated into at least two partitions differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. Following separation, the different partitions can then be subject to further processing steps, which typically include further amplification, and sequence analysis, in parallel but separately. Sequence data from the different partitions can then be compared.

Nucleic acids can be linked at both ends to Y-shaped adapters including primer binding sites and tags. The molecules are amplified. The amplified molecules are then fractionated by contact with an antibody preferentially binding to 5-methylcytosine to produce two partitions. One partition includes original molecules lacking methylation and amplification copies having lost methylation. The other partition includes original DNA molecules with methylation. The two partitions are then processed and sequenced separately with further amplification of the methylated partition. The sequence data of the two partitions can then be compared. In this example, tags are not used to distinguish between methylated and unmethylated DNA but rather to distinguish between different molecules within these partitions so that one can determine whether reads with the same start and stop points are based on the same or different molecules.

The disclosure provides further methods for analyzing a population of nucleic acid in which at least some of the nucleic acids include one or more modified cytosine residues, such as 5-methylcytosine and any of the other modifications described previously. In these methods, the population of nucleic acids is contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are treated with bisulfite. This treatment converts unmodified cytosines to uracils. The bisulfite treated nucleic acids are then subjected to amplification primed by primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Partitioning the Sample into a Plurality of Subsamples; Aspects of Samples; Analysis of Epigenetic Characteristics

In certain embodiments described herein, a population of different forms of nucleic acids (e.g., hypermethylated and hypomethylated DNA in a sample, such as a captured set of cfDNA as described herein) can be physically partitioned based on one or more characteristics of the nucleic acids prior to further analysis, e.g., differentially modifying or isolating a nucleobase, tagging, and/or sequencing. This approach can be used to determine, for example, whether certain sequences are hypermethylated or hypomethylated. In some embodiments, hypermethylation variable epigenetic target regions are analyzed to determine whether they show hypermethylation characteristic of tumor cells and/or hypomethylation variable epigenetic target regions are analyzed to determine whether they show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some instances, a heterogeneous nucleic acid sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein) and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced. In some embodiments, a population of different nucleic acids is partitioned into two or more different partitions. Each partition is representative of a different nucleic acid form, and a first partition (also referred to as a subsample) includes DNA with a cytosine modification in a greater proportion than a second subsample. Each partition is distinctly tagged. The first subsample is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The tagged nucleic acids are pooled together prior to sequencing. Sequence reads are obtained and analyzed, including to distinguish the first nucleobase from the second nucleobase in the DNA of the first subsample, in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine. The affinity agents can be antibodies with the desired specificity, natural binding partners or variants thereof (Bock et al., Nat Biotech 28: 1106-1114 (2010); Song et al., Nat Biotech 29: 68-72 (2011)), or artificial peptides selected e.g., by phage display to have specificity to a given target.

Examples of capture moieties contemplated herein include methyl binding domain (MBDs) and methyl binding proteins (MBPs) as described herein, including proteins such as MeCP2 and antibodies preferentially binding to 5-methylcytosine. Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4, RbAp48 and SANT domain peptides. Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (ThermoFisher Scientific)). Subsequently, additional partitioning may involve eluting fragments having different levels of methylation by adjusting the salt concentration in a solution with the methyl-binding domain and bound fragments. As salt concentration increases, fragments having greater methylation levels are eluted. In some instances, the final partitions are representative of nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MethylMiner Methylated DNA Enrichment Kit (ThermoFisher Scientific) various levels of methylation can be partitioned using sequential elutions. For example, a hypomethylated partition (e.g., no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, at least 300 mM, at least 400 mM, at least 500 mM, at least 600 mM, at least 700 mM, at least 800 mM, at least 900 mM, at least 1000 mM, or at least 2000 mM. After such methylated nucleic acids are eluted, magnetic separation is once again used to separate higher levels of methylated nucleic acids from those with lower level of methylation. The elution and magnetic separation steps can repeat themselves to create various partitions such as a hypomethylated partition (representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent). The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition. For further details regarding portioning nucleic acid samples based on characteristics such as methylation, see WO2018/119452, which is incorporated herein by reference. In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

An exemplary method for molecular tag identification of MBD-bead partitioned libraries through NGS is as follows:

Physical partitioning of an extracted DNA sample (e.g., extracted blood plasma DNA from a human sample) using a methyl-binding domain protein-bead purification kit, saving all elutions from process for downstream processing.

Parallel application of differential molecular tags and NGS-enabling adapter sequences to each partition. For example, the hypermethylated, residual methylation (‘wash’), and hypomethylated partitions are ligated with NGS-adapters with molecular tags.

Re-combining all molecular tagged partitions, and subsequent amplification using adapter-specific DNA primer sequences.

Enrichment/hybridization of re-combined and amplified total library, targeting genomic regions of interest (e.g., cancer-specific genetic variants and differentially methylated regions).

Re-amplification of the enriched total DNA library, appending a sample tag. Different samples are pooled and assayed in multiplex on an NGS instrument.

Bioinformatics analysis of NGS data, with the molecular tags being used to identify unique molecules, as well deconvolution of the sample into molecules that were differentially MBD-partitioned. This analysis can yield information on relative 5-methylcytosine for genomic regions, concurrent with standard genetic sequencing/variant detection.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formylcytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nM to about 2500 mM NaCl. In one embodiment, the process results in three (3) partitions. Molecules are contacted with a solution at a first salt concentration and including a molecule including a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

The disclosure provides further methods for analyzing a population of nucleic acids in which at least some of the nucleic acids include one or more modified cytosine residues, such as 5-methylcytosine and any of the other modifications described previously. In these methods, after partitioning, the subsamples of nucleic acids are contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. The nucleic acids subjected to the procedure are then amplified with primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Such an analysis can be performed using the following exemplary procedure. After partitioning, methylated DNA is linked to Y-shaped adapters at both ends including primer binding sites and tags. The cytosines in the adapters are modified at the 5 position (e.g., 5-methylated). The modification of the adapters serves to protect the primer binding sites in a subsequent conversion step (e.g., bisulfite treatment, TAP conversion, or any other conversion that does not affect the modified cytosine but affects unmodified cytosine). After attachment of adapters, the DNA molecules are amplified. The amplification product is split into two aliquots for sequencing with and without conversion. The aliquot not subjected to conversion can be subjected to sequence analysis with or without further processing. The other aliquot is subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. Only primer binding sites protected by modification of cytosines can support amplification when contacted with primers specific for original primer binding sites. Thus, only original molecules and not copies from the first amplification are subjected to further amplification. The further amplified molecules are then subjected to sequence analysis. Sequences can then be compared from the two aliquots. As in the separation scheme discussed above, nucleic acid tags in adapters are not used to distinguish between methylated and unmethylated DNA but to distinguish nucleic acid molecules within the same partition.

Subjecting the First Subsample to a Procedure that Affects a First Nucleobase in the DNA Differently from a Second Nucleobase in the DNA of the First Subsample

Methods disclosed herein comprise a step of subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases includes mC and the other includes hmC.

In some embodiments, the procedure that affects a first nucleobase in the DNA differently from a second nucleobase in the DNA of the first subsample includes bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase includes one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion on a first subsample as described herein thus facilitates identifying positions containing mC or hmC using the sequence reads obtained from the first subsample. For an exemplary description of bisulfite conversion, see, e.g., Moss et al., Nat Commun. 2018; 9: 5068.

In some embodiments, the procedure that affects a first nucleobase in the DNA differently from a second nucleobase in the DNA of the first subsample includes oxidative bisulfite (Ox-BS) conversion. In some embodiments, the procedure that affects a first nucleobase in the DNA differently from a second nucleobase in the DNA of the first subsample includes Tet-assisted bisulfite (TAB) conversion. In some embodiments, the procedure that affects a first nucleobase in the DNA differently from a second nucleobase in the DNA of the first subsample includes Tet-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes chemical-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes APOBEC-coupled epigenetic (ACE) conversion.

In some embodiments, procedure that affects a first nucleobase in the DNA differently from a second nucleobase in the DNA of the first subsample includes enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-βGT can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA of the first subsample includes separating DNA originally including the first nucleobase from DNA not originally including the first nucleobase.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

Techniques including methylated DNA immunoprecipitation (MeDIP) can be used to separate DNA containing modified bases such as mA from other DNA. See, e.g., Kumar et al., Frontiers Genet. 2018; 9: 640; Greer et al., Cell 2015; 161: 868-878. An antibody specific for mA is described in Sun et al., Bioessays 2015; 37:1155-62. Antibodies for various modified nucleobases, such as forms of thymine/uracil including halogenated forms such as 5-bromouracil, are commercially available. Various modified bases can also be detected based on alterations in their base-pairing specificity. For example, hypoxanthine is a modified form of adenine that can result from deamination and is read in sequencing as a G. See, e.g., U.S. Pat. No. 8,486,630;

Brown, Genomes, 2nd Ed., John Wiley & Sons, Inc., New York, N.Y., 2002, chapter 14, “Mutation, Repair, and Recombination.”

### Enriching/Capturing Step, Amplification, Adaptors, Barcodes

In some embodiments, methods disclosed herein comprise a step of capturing one or more sets of target regions of DNA, such as cfDNA. Capture may be performed using any suitable approach known in the art. In some embodiments, capturing includes contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. Where the first subsample undergoes a separation step (e.g., separating DNA originally including the first nucleobase (e.g., hmC) from DNA not originally including the first nucleobase, such as hmC-seal), capturing may be performed on any, any two, or all of the DNA originally including the first nucleobase (e.g., hmC), the DNA not originally including the first nucleobase, and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein includes capturing cfDNA obtained from a test subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules, and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, see WO2020/160414, which is incorporated herein by reference for all purposes.

In some embodiments, a method described herein includes contacting cfDNA obtained from a test subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test fsor perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In various embodiments, the methods further comprise sequencing the captured cfDNA, e.g., to different degrees of sequencing depth for the epigenetic and sequence-variable target region sets, consistent with the discussion herein. In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some such embodiments, the capturing step is performed with the probes for the sequence-variable target region set and the probes for the epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions including captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, the DNA is amplified. In some embodiments, amplification is performed before the capturing step. In some embodiments, amplification is performed after the capturing step.

In some embodiments, adapters are included in the DNA. This may be done concurrently with an amplification procedure, e.g., by providing the adapters in a 5′ portion of a primer, e.g., as described above. Alternatively, adapters can be added by other approaches, such as ligation.

In some embodiments, tags, which may be or include barcodes, are included in the DNA. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described above. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed in the “General Features of the Methods” section below, which can be combined to the extent practicable with any of the foregoing embodiments and the embodiments set forth in the introduction and summary section.

### Computer Systems, Processing of Real World Evidence (RWE)

Methods of the present disclosure can be implemented using, or with the aid of, computer systems. For example, such methods may comprise: partitioning the sample into a plurality of subsamples, including a first subsample and a second subsample, wherein the first subsample includes DNA with a cytosine modification in a greater proportion than the second subsample; subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity; and sequencing DNA in the first subsample and DNA in the second subsample in a manner that distinguishes the first nucleobase from the second nucleobase in the DNA of the first subsample.

In an aspect, the present disclosure provides a non-transitory computer-readable medium including computer-executable instructions which, when executed by at least one electronic processor, perform at least a portion of a method including: collecting cfDNA from a test subject; capturing a plurality of sets of target regions from the cfDNA, wherein the plurality of target region sets includes a sequence-variable target region set and an epigenetic target region set, whereby a captured set of cfDNA molecules is produced; sequencing the captured cfDNA molecules, wherein the captured cfDNA molecules of the sequence-variable target region set are sequenced to a greater depth of sequencing than the captured cfDNA molecules of the epigenetic target region set; obtaining a plurality of sequence reads generated by a nucleic acid sequencer from sequencing the captured cfDNA molecules; mapping the plurality of sequence reads to one or more reference sequences to generate mapped sequence reads; and processing the mapped sequence reads corresponding to the sequence-variable target region set and to the epigenetic target region set to determine the likelihood that the subject has cancer.

The code can be pre-compiled and configured for use with a machine with a processer adapted to execute the code or can be compiled during runtime. The code can be supplied in a programming language that can be selected to enable the code to execute in a pre-compiled or as-compiled fashion.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7th Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety. Further information is found in PCT Pub. No. US2022032250 and U.S. application Ser. No. 17/832,498.

Described herein is a method to generate an integrated data repository and/or analysis system that includes multiple types of healthcare data, according to one or more implementations. The architecture may include a data integration and/or analysis system. The data integration and analysis system may obtain data from a number of data sources and integrate the data from the data sources into an integrated data repository. For example, the data integration and analysis system may obtain data from a health insurance claims data repository. In various examples, the data integration and analysis system and the health insurance claims data repository may be created and maintained by different entities. In one or more additional examples, the data integration and analysis system and the health insurance claims data repository may be created and maintained by the same entity.

The data integration and analysis system may be implemented by one or more computing devices. The one or more computing devices may include one or more server computing devices, one or more desktop computing devices, one or more laptop computing devices, one or more tablet computing devices, one or more mobile computing devices, or combinations thereof. In certain implementations, at least a portion of the one or more computing devices may be implemented in a distributed computing environment. For example, at least a portion of the one or more computing devices may be implemented in a cloud computing architecture. In scenarios where the computing systems used to implement the data integration and analysis system are configured in a distributed computing architecture, processing operations may be performed concurrently by multiple virtual machines. In various examples, the data integration and analysis system may implement multithreading techniques. The implementation of a distributed computing architecture and multithreading techniques cause the data integration and analysis system to utilize fewer computing resources in relation to computing architectures that do not implement these techniques.

The health insurance claims data repository may store information obtained from one or more health insurance companies that corresponds to insurance claims made by subscribers of the one or more health insurance companies. The health insurance claims data repository may be arranged (e.g., sorted) by patient identifier. The patient identifier may be based on the patient's first name, last name, date of birth, social security number, address, employer, and the like. The data stored by the health insurance claims data repository may include structured data that is arranged in one or more data tables. The one or more data tables storing the structured data may include a number of rows and a number of columns that indicate information about health insurance claims made by subscribers of one or more health insurance companies in relation to procedures and/or treatments received by the subscribers from healthcare providers. At least a portion of the rows and columns of the data tables stored by the health insurance claims data repository may include health insurance codes that may indicate diagnoses of biological conditions, and treatments and/or procedures obtained by subscribers of the one or more health insurance companies. In various examples, the health insurance codes may also indicate diagnostic procedures obtained by individuals that are related to one or more biological conditions that may be present in the individuals. In one or more examples, a diagnostic procedure may provide information used in the detection of the presence of a biological condition. A diagnostic procedure may also provide information used to determine a progression of a biological condition. In one or more illustrative examples, a diagnostic procedure may include one or more imaging procedures, one or more assays, one or more laboratory procedures, one or more combinations thereof, and the like.

The data integration and analysis system may also obtain information from a molecular data repository. The molecular data repository may store data of a number of individuals related to genomic information, genetic information, metabolomic information, transcriptomic information, fragmentomic information, immune receptor information, methylation information, epigenomic information, and/or proteomic information. In one or more examples, the data integration and analysis system and the molecular data repository may be created and maintained by different entities. In one or more additional examples, the data integration and analysis system and the molecular data repository may be created and maintained by a same entity.

The genomic and/or epigenomic information may indicate one or more mutations corresponding to genes of the individuals. A mutation to a gene of individuals may correspond to differences between a sequence of nucleic acids of the individuals and one or more reference genomes. The reference genome may include a known reference genome, such as hg19. In various examples, a mutation of a gene of an individual may correspond to a difference in a germline gene of an individual in relation to the reference genome. In one or more additional examples, the reference genome may include a germline genome of an individual. In one or more further examples, a mutation to a gene of an individual may include a somatic mutation. Mutations to genes of individuals may be related to insertions, deletions, single nucleotide variants, loss of heterozygosity, duplication, amplification, translocation, fusion genes, or one or more combinations thereof.

In one or more illustrative examples, genomic and/or epigenomic information stored by the molecular data repository may include genomic and/or epigenomic profiles of tumor cells present within individuals. In these situations, the genomic and/or epigenomic information may be derived from an analysis of genetic material, such as deoxyribonucleic acid (DNA) and/or ribonucleic acid (RNA) from a sample, including, but not limited to, a tissue sample or tumor biopsy, circulating tumor cells (CTCs), exosomes or efferosomes, or from circulating nucleic acids (e.g., cell-free DNA) found in blood samples of individuals that is present due to the degradation of tumor cells present in the individuals. In one or more examples, the genomic and/or epigenomic information of tumor cells of individuals may correspond to one or more target regions. One or more mutations present with respect to the one or more target regions may indicate the presence of tumor cells in individuals. The genomic and/or epigenomic information stored by the molecular data repository may be generated in relation to an assay or other diagnostic test that may determine one or more mutations with respect to one or more target regions of the reference genome.

The number of data tables may be arranged according to a data repository schema. In the illustrative example of the data repository schema includes a first data table, a second data table, a third data table, a fourth data table, and a fifth data table. Although the illustrative example of includes five data tables, in additional implementations, the data repository schema may include more data tables or fewer data tables. The data repository schema may also include links between the data tables. The links between the data tables may indicate that information retrieved from one of the data tables results in additional information stored by one or more additional data tables to be retrieved. Additionally, not all the data tables may be linked to each of the other data tables. In the illustrative example of the first data table is logically coupled to the second data table by a first link and the first data table is logically coupled to the fourth data table by a second link. In addition, the second data table is logically coupled to the third data table via a third link and the fourth data table is logically coupled to the fifth data table via a fourth link. Further, the third data table is logically coupled to the fifth data table via a fifth link.

In various examples, as data tables are added to and/or removed from the data repository schema, additional links between data tables may be added to or removed from the data repository schema. In one or more illustrative examples, the integrated data repository may store data tables according to the data repository schema for at least a portion of the individuals for which the data integration system obtained information from a combination of at least two of the health insurance claims data repository, the molecular data repository, the one or more additional data repositories, and the one or more reference information data repositories. As a result, the integrated data repository may store respective instances of the data tables according to the data repository schema for thousands, tens of thousands, up to hundreds of thousands or more individuals.

The data integration and analysis system may also include a data pipeline system. The data pipeline system may include a number of algorithms, software code, scripts, macros, or other bundles of computer-executable instructions that process information stored by the integrated data repository to generate additional datasets. The additional datasets may include information obtained from one or more of the data tables. The additional datasets may also include information that is derived from data obtained from one or more of the data tables. The components of the data pipeline system implemented to generate a first additional dataset may be different from the components of the data pipeline system used to generate a second additional dataset.

In one or more examples, the data pipeline system may generate a dataset that indicates pharmacy treatments received by a number of individuals. In one or more illustrative examples, the data pipeline system may analyze information stored in at least one of the data tables to determine health insurance codes corresponding to pharmaceutical treatments received by a number of individuals. The data pipeline system may analyze the health insurance codes corresponding to pharmaceutical treatments with respect to a library of data that indicates specified pharmaceutical treatments that correspond to one or more health insurance codes to determine names of pharmaceutical treatments that have been received by the individuals. In one or more additional examples, the data pipeline system may analyze information stored by the integrated data repository to determine medical procedures received by a number of individuals. To illustrate, the data pipeline system may analyze information stored by one of the data tables to determine treatments received by individuals via at least one injection or intravenously. In one or more further examples, the data pipeline system may analyze information stored by the integrated data repository to determine episodes of care for individuals, lines of therapy received by individuals, progression of a biological condition, or time to next treatment. In various examples, the datasets generated by the data pipeline system may be different for different biological conditions. For example, the data pipeline system may generate a first number of datasets with respect to a first type of cancer, such as lung cancer, and a second number of datasets with respect to a second type of cancer, such as colorectal cancer.

The data pipeline system may also determine one or more confidence levels to assign to information associated with individuals having data stored by the integrated data repository. The respective confidence levels may correspond to different measures of accuracy for information associated with individuals having data stored by the integrated data repository. The information associated with the respective confidence levels may correspond to one or more characteristics of individuals derived from data stored by the integrated data repository. Values of confidence levels for the one or more characteristics may be generated by the data pipeline system in conjunction with generating one or more datasets from the integrated data repository. In one or more examples, a first confidence level may correspond to a first range of measures of accuracy, a second confidence level may correspond to a second range of measures of accuracy, and a third confidence level may correspond to a third range of measures of accuracy. In one or more additional examples, the second range of measures of accuracy may include values that are less values of the first range of measures of accuracy and the third range of measures of accuracy may include values that are less than values of the second range of measures of accuracy. In one or more illustrative examples, information corresponding to the first confidence level may be referred to as Gold standard information, information corresponding to the second confidence level may be referred to as Silver standard information, and information corresponding to the third confidence level may be referred to as Bronze standard information.

The data pipeline system may determine values for the confidence levels of characteristics of individuals based on a number of factors. For example, a respective set of information may be used to determine characteristics of individuals. The data pipeline system may determine the confidence levels of characteristics of individuals based on an amount of completeness of the respective set of information used to determine a characteristic for an individual. In situations where one or more pieces of information are missing from the set of information associated with a first number of individuals, the confidence levels for a characteristic may be lower than for a second number of individuals where information is not missing from the set of information. In one or more examples, an amount of missing information may be used by the data pipeline system to determine confidence levels of characteristics of individuals. To illustrate, a greater amount of missing information used to determine a characteristic of an individual may cause confidence levels for the characteristic to be lower than in situations where the amount of missing information used to determine the characteristic is lower. Further, different types of information may correspond to various confidence levels for a characteristic. In one or more examples, the presence of a first piece of information used to determine a characteristic of an individual may result in confidence levels for the characteristic being higher than the presence of a second piece of information used to determine the characteristic.

In one or more illustrative examples, the data pipeline system may determine a number of individuals included in a cohort with a primary diagnosis of lung cancer (or other biological condition). The data pipeline system may determine confidence levels for respective individuals with respect to being classified as having a primary diagnosis of lung cancer. The data pipeline system may use information from a number of columns included in the data tables to determine a confidence level for the inclusion of individuals within a lung cancer cohort. The number of columns may include health insurance codes related to diagnosis of biological conditions and/or treatments of biological conditions. Additionally, the number of columns may correspond to dates of diagnosis and/or treatment for biological conditions. The data pipeline system may determine that a confidence level of an individual being characterized as being part of the lung cancer cohort is higher in scenarios where information is available for each of the number of columns or at least a threshold number of columns than in instances where information is available for less than a threshold number of columns. Further, the data pipeline system may determine confidence levels for individuals included in a lung cancer cohort based on the type of information and availability of information associated with one or more columns. To illustrate, in situations where one or more diagnosis codes are present in relation to one or more periods of time for a group of individuals and one or more treatment codes are absent, the data pipeline system may determine that the confidence level of including the group of individuals in the lung cancer cohort is greater than in situations where at least one of the diagnosis codes is absent and the treatment codes used to determine whether individuals are included in the lung cancer cohort are present.

The data analysis system may receive integrated data repository requests from one or more computing devices, such as an example computing device. The one or more integrated data repository requests may cause data to be retrieved from the integrated data repository. In various examples, the one or more integrated data repository requests may cause data to be retrieved from one or more datasets generated by the data pipeline system. The integrated data repository requests may specify the data to be retrieved from the integrated data repository and/or the one or more datasets generated by the data pipeline system. In one or more additional examples, the integrated data repository requests may include one or more prebuilt queries that correspond to computer-executable instructions that retrieve a specified set of data from the integrated data repository and/or one or more datasets generated by the data pipeline system.

In response to one or more integrated data repository requests, the data analysis system may analyze data retrieved from at least one of the integrated data repository or one or more datasets generated by the data pipeline system to generate data analysis results. The data analysis results may be sent to one or more computing devices, such as example computing devices. Although the illustrative example of hows that the one or more integrated data repository requests from one computing device and the data analysis results being sent to another computing device, in one or more additional implementations, the data analysis results may be received by a same computing device that sent the one or more integrated data repository requests. The data analysis results may be displayed by one or more user interfaces rendered by the computing device or the computing device.

Described herein is a method for analysis of nucleic acid sequence information. In various embodiments, the method of analysis comprises one or more models, each of one or more including one or more of survival, sub-modeling, disease node determination and identification (e.g., driver mutations), disease association, disease subtyping, recurrence, metastasis, time to next treatment, etc. as separate components. In various embodiments, a model includes hierarchal models (e.g., nested models, multi-level models), mixed models (e.g., regression such as logistic regression and Poisson regression, pooled, random effect, fixed effect, mixed effect, linear mixed effect, generalized linear mixed effect), hazard model, odds ratio models and/or repeated sample (e.g., repeated measures such as ANOVA). In various embodiments, the model is a hierarchical random effects model. In various embodiments, the model is a hierarchical cubic spline random effects model. In various embodiments, the model is a cubic spline model. In various embodiments, the model is a generalized linear effects model. In various embodiments, the model is a linear effects model. In various embodiments, the model is a Cox proportional hazard model. In various embodiments, the method of analysis comprises assembly of models together. In various embodiments assembly includes generation of association parameters. In one or more embodiments, the method of analysis includes patient survival information and patient genetic information. As an example, assembly of models together could include different models for the different types of cancers, including subtypes, represented in the patient survival information. Each of different models can be configured to determine correlations between genetic factors and the survival times of patients diagnosed with the respective types of cancers they are configured to evaluate. For example, genetic factors determined to have strong correlations to cancer survival times (e.g., relatively short survival times and/or relatively long survival times) can be recommended as potential therapeutic targets.

In various embodiments, analysis can include one or more of survival, sub-modeling, disease node determination and identification (e.g., driver mutations), disease association, disease subtyping, recurrence, metastasis, time to next treatment, etc. as separate components. For example modeling can facilitate applying the aforementioned, such as patient survival information and the patient genetic information. In various embodiments, a sub-modeling component can determines subsets of the patient survival information and the patient genetic information for generations different patient cohorts associated with different types of cancer and cancer subtypes. In various embodiments, a sub-model includes hierarchal models (e.g., nested models, multi-level models), mixed models (e.g., regression such as logistic regression and Poisson regression, pooled, random effect, fixed effect, mixed effect, linear mixed effect, generalized linear mixed effect), hazard model, odds ratio models and/or repeated sample (e.g., repeated measures such as ANOVA). In various embodiments, the sub-model is a hierarchical random effects model. In various embodiments, the sub-model is a hierarchical cubic spline random effects model. In various embodiments, the sub-model is a cubic spline model. In various embodiments, the sub-model is a generalized linear effects model. In various embodiments, the sub-model is a linear effects model. In various embodiments, the sub-model is a Cox proportional hazard model. Each subset of the patient survival information and the patient genetic information can comprise information for patients diagnosed with a different type of cancer and cancer subtypes. For example, the sub-modeling component can further apply the subsets of the patient survival information and the patient genetic information to corresponding individual survival models developed for the different cancer types, including subtypes. In various embodiments, information generated for the method of analysis can be stored in memory (e.g., as model data). In various embodiments, and information generated for the method of analysis generates one or more survival models for individual subjects.

In various embodiments, analysis of the patient survival information and the patient genetic information using the survival models, include the disease node determination and identification component can identify, for each type of cancer, disease nodes included in the patient genetic information that are involved in the genetic mechanisms employed by the respective cancer types to proliferate. In various embodiments, the disease node component identifies disease node based on observed correlations between genetic factors and the cancer survival times provided in the patient survival information. For example, a genetic factor that is frequently observed in association with short survival times of a specific type of cancer and less frequently observed in association with long survival times of the specific type of cancer can be identified as an active genetic factor having an active role in the genetic mechanism of the specific type of cancer, including subtypes.

In various embodiments, disease node determination and identification includes disease association parameters regarding associations between different cancer types to facilitate identifying the active genetic factors associated with the different cancer types. For example, cancer types which are highly associated can share one or more common critical underlying genetic factors. As readily appreciated by one of ordinary skill, models (e.g., survival model) of associated cancer types dialectically exchange information to determine and/or identify active genetic factors across types of cancer, including subtypes. In various embodiments, the disease association parameters applied by the disease node determination and identification is facilitated by modeling. In various embodiments, generation of individual survival models can employ one or more machine learning algorithms to facilitate the determination and/or identification of the survival, modeling, disease node associated with a particular type of cancer, including subtypes, based on the, and the patient genetic information and the disease association parameters.

In some embodiments, in association with node determination and identification for cancer type, including subtypes, includes determination of a score system for the disease node(s). For example, a score for a disease node with respect to a specific type of cancer, including subtypes, reflects association of the disease node to the survival time of the specific type of cancer, including subtypes. In various embodiments, scores can be based on a frequency with which a particular genetic factor is directly or indirectly identified for patients diagnosed with a specific cancer type. In various embodiments, analysis includes the aforementioned survival, sub-modeling, disease node determination and identification (e.g., driver mutations), disease association, disease subtyping, recurrence, metastasis, time to next treatment, etc. can be related to, less than a defined threshold, greater than a defined threshold. For example, greater scores associated with a disease node and cancer type, including, the greater the contribution of the disease node to survival time. In various embodiments, formation regarding disease nodes for respective types of cancer, including subtypes and scores determined for the active genetic factors can be collated in a data structure, such as a database.

Described herein is a method of analysis that includes effects modeling. In various embodiments, the effects modeling includes random effect, fixed effect, mixed effect, linear mixed effect, and generalized linear mixed effect. In various embodiments, the effects includes cubic spline. In various embodiments, the effects modeling includes regression. In various embodiments, the effects modeling includes logistic and Poisson regression. In various embodiments, the model does not include covariates. In In various embodiments, the model includes covariates. In various embodiments, the covariates are information from medical records (including laboratory testing records such as genomic, epigenomic, nucleic acid and other analyte results), insurance records or the like. Examples include age, line of therapies, smoking status (yes/no), gender, and various scoring and/or staging systems that have been utilized for specific cancer disease patients, with an illustrative example including age (in years), line of anti-EGFR therapy, smoking status (yes/no), gender (female/male), and the Van Walraven Elixhauser Comorbidity (ELIX) score specific to lung cancer patients (expressed as a weighted measure across multiple common comorbidities. One of skill readily appreciates covariates can include any number of data elements for individuals and individuals in a population, such as that from medical records (including laboratory testing records such as genomic, epigenomic, nucleic acid and other analyte results), insurance records or the like.

In various embodiments, the method of analysis includes generation of a hierarchy including at least one first level equation. In various embodiments, a first level equation includes a truncated cubic spline. In various embodiments, the truncated cubic spline includes longitudinal data. This includes, for example, direct or indirect measurements of ctDNA levels, allele fractions, tumor fractions. In various embodiments, an additional level equation includes covariate. In various embodiments, the covariates are information for an individual, or individuals in a population, drawn and/or stored from medical records (including laboratory testing records such as genomic, epigenomic, nucleic acid and other analyte results), insurance records, or the like. Examples include age, line of therapies, smoking status (yes/no), gender, and various scoring and/or staging systems that have been utilized for specific cancer disease patients. In various embodiments, a velocity plot is generated. In various embodiments, the velocity plot is a derivative or one or equations, such as at least one first level equation. In various embodiments, the method of analysis includes one or more of Equations (1), (2) and (3) described in the Examples.

Described herein is a method of analysis that includes jointly solving different analysis components, including one or more of survival, modeling and sub-modeling, disease node determination and identification (e.g., driver mutations), disease association, disease subtyping, recurrence, metastasis, time to next treatment, etc. as separate components. In various embodiments, the method of analysis includes jointly solving one or more different models for the different cancer types under a joint model framework. For example, the method of analysis could include jointly solving one or more different survival models for the different cancer types under a joint model framework. In various embodiments, the method includes determination of association parameters. In various embodiments, association parameters include, for example, the relationship between patient survival and the patient's estimated current value of the biomarker, the relationship between patient survival and patient's estimated current change over time with respect to the biomarker. In various embodiments, this includes the slope, and the relationship between overall survival and current estimated area under a subject's longitudinal trajectory as a surrogate for a biomarker's cumulative effect. It is readily appreciated by one of ordinary skill that association parameters can undertake multiple forms, and can also be combined. For instance, one could examine the relationship between overall survival and estimated current value plus the estimated current slope of the patient's longitudinal trajectory.

In one or more examples, the data analysis system may implement at least one of one or more machine learning techniques or one or more statistical techniques to analyze data retrieved in response to one or more integrated data repository requests. In one or more examples, the data analysis system may implement one or more artificial neural networks to analyze data retrieved in response to one or more integrated data repository requests. To illustrate, the data analysis system may implement at least one of one or more convolutional neural networks or one or more residual neural networks to analyze data retrieved from the integrated data repository in response to one or more integrated data repository requests. In at least some examples, the data analysis system may implement one or more random forests techniques, one or more support vector machines, or one or more Hidden Markov models to analyze data retrieved in response to one or more integrated data repository requests. One or more statistical models may also be implemented to analyze data retrieved in response to one or more integrated data repository requests to identify at least one of correlations or measures of significance between characteristics of individuals. For example, log rank tests may be applied to data retrieved in response to one or more integrated data repository requests. In addition, Cox proportional hazards models may be implemented with respect to date retrieved in response to one or more integrated data repository requests. Further, Wilcoxon signed rank tests may be applied to data retrieved in response to one or more integrated data repository requests. In still other examples, a z-score analysis may be performed with respect to data retrieved in response to one or more integrated data repository requests. In still additional examples, a Kaplan Meier analysis may be performed with respect to data retrieved in response to one or more integrated data repository requests. In at least some examples, one or more machine learning techniques may be implemented in combination with one or more statistical techniques to analyze data retrieved in response to one or more integrated data repository requests.

In one or more illustrative examples, the data analysis system may determine a rate of survival of individuals in which lung cancer is present in response to one or more treatments. In one or more additional illustrative examples, the data analysis system may determine a rate of survival of individuals having one or more genomic and/or epigenomic region mutations in which lung cancer is present in response to one or more treatments. In various examples, the data analysis system may generate the data analysis results in situations where the data retrieved from at least one of the integrated data repository or the one or more datasets generated by the data pipeline system satisfies one or more criteria. For example, the data analysis system may determine whether at least a portion of the data retrieved in response to one or more integrated data repository requests satisfies a threshold confidence level. In situations where the confidence level for at least a portion of the date retrieved in response to one or more integrated data repository requests is less than a threshold confidence level, the data analysis system may refrain from generating at least a portion of data analysis results. In scenarios where the confidence level for at least a portion of the data retrieved in response to one or more integrated data repository requests is at least a threshold confidence level, the data analysis system may generate at least a portion of the data analysis results. In various examples, the threshold confidence level may be related to the type of data analysis results being generated by the data analysis system.

In one or more illustrative examples, the data analysis system may receive an integrated data repository request to generate data analysis results that indicate a rate of survival of one or more individuals. In these instances, the data analysis system may determine whether the data stored by the integrated data repository and/or by one or more datasets generated by the data pipeline system satisfies a threshold confidence level, such as a Gold standard confidence level. In one or more additional examples, the data analysis system may receive an integrated data repository request to generate data analysis results that indicate a treatment received by one or more individuals. In these implementations, the data analysis system may determine whether the data stored by the integrated data repository and/or by one or more datasets generated by the data pipeline system satisfies a lower threshold confidence level, such as a Bronze standard confidence level.

In one or more additional illustrative examples, the data analysis system may receive an integrated data repository request to determine individuals having one or more genomic and/or epigenomic mutations and that have received one or more treatments for a biological condition. Continuing with this example, the data analysis system can determine a survival rate of individuals with the one or more genomic and/or epigenomic mutations in relation to the one or more treatments received by the individuals. The data analysis system can then identify based on the survival rate of individuals and effectiveness of treatments for the individuals in relation to genomic and/or epigenomic mutations that may be present in the individuals. In this way, health outcomes of individuals may be improved by identifying prospective treatments that may be more effective for populations of individuals having one or more genomic and/or epigenomic mutations than current treatments being provided to the individuals.

The data pipeline system may include first data processing instructions, second data processing instructions, up to Nth data processing instructions. The data processing instructions, may be executable by one or more processing units to perform a number of operations to generate respective datasets using information obtained from the integrated data repository. In one or more illustrative examples, the data processing instructions, may include at least one of software code, scripts, API calls, macros, and so forth. The first data processing instructions may be executable to generate a first dataset. In addition, the second data processing instructions may be executable to generate a second dataset. Further, the Nth data processing instructions may be executable to generate an Nth dataset. In various examples, after the data integration and analysis system generates the integrated data repository, the data pipeline system may cause the data processing instructions, to be executed to generate the datasets. In one or more examples, the datasets, may be stored by the integrated data repository or by an additional data repository that is accessible to the data integration and analysis system. At least a portion of the data processing instructions may analyze health insurance codes to generate at least a portion of the datasets. Additionally, at least a portion of the data processing instructions may analyze genomics data to generate at least a portion of the datasets.

In one or more examples, the first data processing instructions may be executable to retrieve data from one or more first data tables stored by the integrated data repository. The first data processing instructions may also be executable to retrieve data from one or more specified columns of the one or more first data tables. In various examples, the first data processing instructions may be executable to identify individuals that have a health insurance code stored in one or more column and row combinations that correspond to one or more diagnosis codes. The first data processing instructions may then be executable to analyze the one or more diagnosis codes to determine a biological condition for which the individuals have been diagnosed. In one or more illustrative examples, the first data processing instructions may be executable to analyze the one or more diagnosis codes with respect to a library of diagnosis codes that indicates one or more biological conditions that correspond to respective diagnosis codes. The library of diagnosis codes may include hundreds up to thousands of diagnosis codes. The first data processing instructions may also be executable to determine individuals diagnosed with a biological condition by analyzing timing information of the individuals, such as dates of treatment, dates of diagnosis, dates of death, one or more combinations thereof, and the like.

The second data processing instructions may be executable to retrieve data from one or more second data tables stored by the integrated data repository. The second data processing instructions may also be executable to retrieve data from one or more specified columns of the one or more second data tables. In various examples, the second data processing instructions may be executable to identify individuals that have a health insurance code stored in one or more columns and row combinations that correspond to one or more treatment codes. The one or more treatment codes may correspond to treatments obtained from a pharmacy. In one or more additional examples, the one or more treatment codes may correspond to treatments received by a medical procedure, such as an injection or intravenously. The second data processing instructions may be executable to determine one or more treatments that correspond to the respective health insurance codes included in the one or more second data tables by analyzing the health insurance code in relation to a predetermined set of information. The predetermined set of information may include a data library that indicates one or more treatments that correspond to one out of hundreds up to thousands of health insurance codes. The second data processing instructions may generate the second dataset to indicate respective treatments received by a group of individuals. In one or more illustrative examples, the group of individuals may correspond to the individuals included in the first dataset. The second dataset may be arranged in rows and columns with one or more rows corresponding to a single individual and one or more columns indicating the treatments received by the respective individual.

The Nth processing instructions (where N may be any positive integer) may be executable to generate the Nth dataset by combining information from a number of previously generated datasets, such as the first dataset and the second dataset. In addition, the Nth processing instructions may be executable to generate the Nth dataset to retrieve additional information from one or more additional columns of the integrated data repository and incorporate the additional information from the integrated data repository with information obtained from the first dataset and the second dataset. For example, the Nth processing instructions may be executable to identify individuals included in the first dataset that are diagnosed with a biological condition and analyze specified columns of one or more additional data tables of the integrated data repository to determine dates of the treatments indicated in the second dataset that correspond to the individuals included in the first dataset. In one or more further examples, the Nth processing instructions may be executable to analyze columns of one or more additional data tables of the integrated data repository to determine dosages of treatments indicated in the second dataset received by the individuals included in the first dataset. In this way, the Nth processing instructions may be executable to generate an episodes of care dataset based on information included in a cohort dataset and a treatments dataset.

In one or more illustrative examples, in response to receiving an integrated data repository request, the data analysis system may determine one or more datasets that correspond to the features of the query related to the integrated data repository request. For example, the data analysis system may determine that information included in the first dataset and the second dataset is applicable to responding to the integrated data repository request. In these scenarios, the data analysis system may analyze at least a portion of the data included in the first dataset and the second dataset to generate the data analysis results. In one or more additional examples, the data analysis system may determine different datasets to respond to different queries included in the integrated data repository request in order to generate the data analysis results.

The use of specific sets of data processing instructions to generate respective data sets may reduce the number of inputs from users of the data integration and analysis system as well as reduce the computational load, such as the amount of processing resources and memory, utilized to process integrated data repository requests. For example, without the specific architecture of the data pipeline system, each time an integrated data repository request is received, the data utilized to respond to the integrated data repository request is assembled from the data repository. In contrast, by implementing the data pipeline system to execute the data processing instruction to generate the datasets the data needed to respond to various integrated data repository requests has already been assembled and may be accessed by the data analysis system to respond to the integrated data repository request. Thus, the computing resources used to respond to the integrated data repository request by implementing the data pipeline system to generate the datasets are less than typical systems that perform an information parsing and collecting process for each integrated data repository request. Further, in situations where the data pipeline system has not been implemented, users of the data integration and analysis system may need to submit multiple integrated data repository request in order to analyze the information that the users are intending to have analyzed either because the ad hoc collection of data to respond to an integrated data repository request in typical systems is inaccurate or because the data analysis system is called upon multiple times to perform an analysis of information in typical systems that may be performed using a single integrated data repository request when the data pipeline system is implemented.

At operation, the data integration and analysis system may integrate genomics data and health insurance claims data of individuals that are common to both the molecular data repository and the health insurance claims data repository. The data integration and analysis system may determine individuals that are common to both the molecular data repository and the health insurance claims data repository by determining genomics data and health insurance claims data corresponding to common tokens. The data integration and analysis system may determine that a first token related to a portion of the genomics data corresponds to a second token related to a portion of the health insurance claims data by determining a measure of similarity between the first token and the second token. In scenarios where the first token has at least a threshold amount of similarity with respect to the second token, the data integration and analysis system may store the corresponding portion of the genomics data and the corresponding portion of the health insurance claims data in relation to the identifier of the individual in an integrated data repository, such as an integrated data repository.

The implementation of the architecture may implement a cryptographic protocol that enables de-identified information from disparate data repositories to be integrated into a single data repository. In this way, the security of the data stored by the integrated data repository is increased. Additionally, the cryptographic protocol implemented by the architecture may enable more efficient retrieval and accurate analysis of information stored by the integrated data repository than in situations where the cryptographic protocol of the architecture is not utilized. For example, by generating a token file that includes first tokens using a cryptographic technique based on a specified set of information stored by the molecular data repository and utilizing second tokens generated using a same or similar cryptographic technique with respect to the similar or same set of information stored by the health insurance claims data repository, the data integration and analysis system may match information stored by disparate data repositories that correspond to a same individual. Without implementing the cryptographic protocol of the architecture, the probability of incorrectly attributing information from one data repository to one or more individuals increases, which decreases the accuracy of results provided by the data integration and analysis system in response to integrated data repository requests sent to the data integration and analysis system.

Described herein is a framework to generate a dataset, by a data pipeline system, based on data stored by an integrated data repository, according to one or more implementations. The integrated data repository may store health insurance claims data and genomics data for a group of individuals. For example, the integrated data repository may store information obtained from health insurance claims records of the group of individuals. For each individual included in the group of individuals, the integrated data repository may store information obtained from multiple health insurance claim records. In various examples, the information stored by the integrated data repository may include and/or be derived from thousands, tens of thousands, hundreds of thousands, up to millions of health insurance claims records for a number of individuals. Additionally, each health insurance claim record may include multiple columns. As a result, the integrated data repository may be generated through the analysis of millions of columns of health insurance claims data.

Further, although the health insurance claims data may be organized according to a structured data format, health insurance claims data is typically arranged to be viewed by health insurance providers, patients, and healthcare providers in order to show financial information and insurance code information related to services provided to individuals by healthcare providers. Thus, health insurance claims data is not easily analyzed to gain insights that may be available in relation to characteristics of individuals in which a biological condition is present and that may aid in the treatment of the individuals with respect to the biological condition. The integrated data repository may be generated and organized by analyzing and modifying raw health insurance claims data in a manner that enables the data stored by the integrated data repository to be further analyzed to determine trends, characteristics, features, and/or insights with respect to individuals in which one or more biological conditions may be present. For example, health insurance codes may be stored in the integrated data repository in such a way that at least one of medical procedures, biological conditions, treatments, dosages, manufacturers of medications, distributors of medications, or diagnoses may be determined for a given individual based on health insurance claims data for the individual. In various examples, the data integration and analysis system may generate and implement one or more tables that indicate correlations between health insurance claims data and various treatments, symptoms, or biological conditions that correspond to the health insurance claims data. Further, the integrated data repository may be generated using genomics data records of the group of individuals. In various examples, the large amounts of health insurance claims data may be matched with genomics data for the group of individuals to generate the integrated data repository.

By integrating the genomics data records for the group of individuals with the health insurance claims records, the data integration and analysis system may determine correlations between the presence of one or more biomarkers that are present in the genomics data records with other characteristics of individuals that are indicated by the health insurance claims data records that existing systems are typically unable to determine. For example, the data integration and analysis system may determine one or more genomic and/or epigenomic characteristics of individuals that correspond to treatments received by individuals, timing of treatments, dosages of treatments, diagnoses of individuals, smoking status, presence of one or more biological conditions, presence of one or more symptoms of a biological condition, one or more combinations thereof, and the like. Based on the correlations determined by the data integration and analysis system using the integrated data repository, cohorts of individuals that may benefit from one or more treatments may be identified that would not have been identified in existing systems. In one or more examples, the processes and techniques implemented to integrate the health insurance claims records and the genomics claims records in order to generate the integrated data repository may be complex and implement efficiency-enhancing techniques, systems, and processes in order to minimize the amount of computing resources used to generate the integrated data repository.

In one or more illustrative examples, the data pipeline system may access information stored by the integrated data repository to generate datasets that include a number of additional data records that include information related to at least a portion of the group of individuals. In an illustrative example, the additional data record includes information indicating whether individuals are included in a cohort of individuals in which lung cancer is present. The data pipeline system may execute a plurality of different sets of data processing instructions to determine a cohort of the group of individuals in which lung cancer is present. In various examples, the additional data record may indicate information used to determine a status of an individual with respect to lung cancer, such as one or more transaction insurance identifier, one or more international classification of diseases (ICD) codes, and one or more health insurance transaction dates. In addition to including a column that indicates whether an individual is included in the lung cancer cohort, the additional data record may include a column indicating a confidence level of the status of the individual with respect to the presence of lung cancer.

Described herein is a computing architecture to incorporate medical records data into an integrated data repository. In various examples, at least a portion of the operations of the computing architecture may be performed by the data integration and analysis system. In one or more examples, at least a portion of the operations of the computing architecture may be performed by one or more additional computing systems that are at least one of controlled, maintained, or implemented by a service provider that also at least one of controls, maintains, or implements the data integration and analysis system. In one or more additional examples, at least a portion of the operations of the computing architecture may be performed by a number of servers in a distributed computing environment.

The computing architecture may include a medical records data repository. The medical records data repository may store medical records data from a number of individuals. The medical records data may include imaging information, laboratory test results, diagnostic test information, clinical observations, dental health information, notes of healthcare practitioners, medical history forms, diagnostic request forms, medical procedure order forms, medical information charts, one or more combinations thereof, and so forth. In various examples, for a given individual, the medical records data repository may store information obtained from one or more healthcare practitioners that is related to the individual.

The computing architecture may perform an operation that includes obtaining data packages from the medical records data repository. In one or more examples, the data packages may be obtained in response to one or more requests sent to the medical records data repository for medical records that correspond to one or more individuals. In one or more additional examples, the data packages may be obtained by the computing architecture using one or more application programming interface (API) calls. In one or more illustrative examples, a first data package, a second data package, up to an Nth data package may be obtained using the computing architecture. The individual data packages, may correspond medical records of a respective individual. For example, the first data package may include medical records of a first individual, the second data package may include medical records of a second individual, and the Nth data package may include medical records of a third individual.

Individual data packages, may include a number of components. In one or more examples, individual data packages, may include individual components that correspond to medical records from different healthcare providers. In one or more additional examples, the individual data packages, may include individual components that correspond to different parts of medical records that correspond to one or more healthcare providers. In an illustrative example the second data package may include a first component, a second component, up to an Nth component. In one or more illustrative examples, the first component may include a first portion of medical records of an individual, the second component may include a second portion of medical records of an individual, and the Nth component may include a third portion of medical records of an individual. In various examples, the first component may correspond to medical records of a first healthcare provider for the individual, the second component may correspond to medical records of a second healthcare provider for the individual, and the third component may correspond to medical records of a third healthcare provider for the individual. In one or more additional illustrative examples, the first component may include a first section of medical records of the individual, such as one or more forms related to a diagnostic test or procedure, and the second component may include a second section of medical records of the individual, such as a pathology report of the individual.

At operation, the computing architecture may preprocess individual data packages to identify a corpus of information to be analyzed. In one or more examples, the preprocessing of data packages obtained from the medical records data repository, may include transforming the data included in the data packages. For example, preprocessing the data packages may include transforming at least a portion of the data obtained from the medical records data repository to machine encoded information. To illustrate, preprocessing the data packages may include performing one or more optical character recognition (OCR) operations with respect to at least a portion of the data packages obtained from the medical records data repository. By converting at least a portion of the data packages obtained from the medical records data repository to machine encoded information, the data packages may be subjected to a number of operations, such as one or more parsing operations to identify one or more characters or strings of characters or one or more editing operations that are unable to be performed with respect to at least a portion of the data packages obtained from the medical records data repository.

In one or more examples, the preprocessing of individual data packages may include determining information included in individual data packages that is to be excluded from further analysis by the computing architecture. In various examples, one or more components of individual data packages may be excluded from a corpus of information to be analyzed. For example, with respect to the second data package, the computing architecture may determine that the first component is to be excluded from further analysis by the computing architecture. In one or more examples, the computing architecture may analyze the components, and/or with respect to one or more keywords to identify at least one of the components, and/or to exclude from further analysis by the computing architecture. In one or more illustrative examples, the computing architecture may parse the components, and/or to identify one or more keywords and in response to identifying the one or more keywords in a component, and/or, the computing architecture may determine to exclude the respective component, and/or from further analysis by the computing architecture. For example, the computing architecture may determine that the first component of the second data package is a test requisition form for one or more diagnostic procedures or tests. In these scenarios, the computing architecture may determine that the first component is to be excluded from further analysis by the computing architecture. Additionally, the computing architecture may determine that at least one of the second component and/or correspond to one or more pathology reports for an individual based on one or more keywords included in at least one of the second component or the Nth component. In these instances, the computing architecture may determine that at least a portion of the second component and/or at least a portion of the Nth component is to be included in the corpus of information to be further analyzed by the computing architecture.

In addition, a subset of the components of individual data packages obtained from the medical records data repository may be included in the corpus of information. In various examples, one or more additional operations may be performed to narrow the corpus of information. For example, one or more queries may be applied to a subset of information obtained from the medical records data repository. The one or more queries may extract information from the one or more data packages that satisfy the one or more queries. In at least some examples, the one or more queries may be a group of queries that are applied to individual components of a data package. In one or more illustrative examples, the group of queries may determine information to be included in the corpus of information and additional information that is to be excluded from the corpus of information. In one or more additional examples, one or more sections of at least one component of a data package may be excluded from the corpus of information.

In one or more additional illustrative examples, after determining that the first component is to be excluded from further analysis by the computing architecture, the computing architecture may then cause one or more queries to be implemented with respect to at least one the second component or the Nth component. In these scenarios, the one or more queries may determine that a section of the second component, such as a section that indicates family history for one or more biological conditions, is to be excluded from the corpus of information. In various examples, the one or more queries may be directed to identifying a number of keywords and/or combinations of keywords included in at least one of the second component or the Nth component. In these instances, the computing architecture may exclude from the corpus of information one or more portions of the individual components of the data packages that include one or more keywords or combinations of keywords. In one or more additional examples, the computing architecture may exclude from the corpus of information a number of words, a number of characters, and/or a number of symbols following one or more keywords that are included in one or more portions of the individual components of the data packages.

Further, at operation, the computing architecture may analyze the corpus of information to determine characteristics of individuals. In one or more examples, the computing architecture may analyze the corpus of information to determine individuals that have one or more phenotypes. In various examples, the computing architecture may analyze the corpus of information to determine one or more biomarkers that are indicative of a biological condition. For example, the computing architecture may analyze the corpus of information to determine individuals having one or more genetic characteristics. The one or more genetic characteristics may include at least one of one or more variants of a genomic and/or epigenomic region that correspond to a biological condition. In one or more illustrative examples, the one or more genetic characteristics may correspond to one or more variants of a genomic and/or epigenomic region that correspond to a type of cancer. In one or more additional illustrative examples, the one or more biomarkers may correspond to levels of an analyte being outside of a specified range. To illustrate, the computing architecture may analyze the corpus of information to determine individuals having levels of one or more proteins and/or levels of one or more small molecules present that are indicative of a biological condition. In these scenarios, the computing architecture may analyze results of laboratory tests to determine levels of analytes of individuals. In one or more additional examples, the computing architecture may analyze the corpus of information to determine individuals in which one or more symptoms are present that are indicative of a biological condition. In one or more further examples, the computing architecture may analyze imaging information included in the corpus of information to determine individuals in which one or more biomarkers are present.

In one or more examples, the computing architecture may implement one or more machine learning techniques to analyze the corpus of information. For example, the computing architecture may implement one or more artificial neural networks, such as at least one of one or more convolutional neural networks or one or more residual neural networks to analyze the corpus of information. The computing architecture may also implement at least one of one or more random forests techniques, one or more hidden Markov models, or one or more support vector machines to analyze the corpus of information.

In at least some implementations, the computing architecture may analyze the corpus of information by performing one or more queries with respect to the corpus of information. The one or more queries may correspond to one or more keywords and/or combinations of keywords. The one or more keywords and/or combinations of keywords may correspond to at least one of characters or symbols that correspond to one or more biological conditions. To illustrate, a keyword may correspond to characters related to a mutation of a genomic and/or epigenomic region, such as HER2. In one or more additional illustrative examples, one or more criteria may be associated with combinations of keyworks. To illustrate, a criterion that corresponds to a combination of keywords may include a number of words being present within a specified distance of one another in a portion of the corpus of information for an individual, such as the words fatigue, blood pressure, and swelling occurring within characters of one another. In these instances, the computing architecture may parse the corpus of information for the one or more keywords and/or combinations of keywords. In various examples, in response to determining that the one or more keywords and/or combinations of keywords are present in accordance with one or more criteria, the computing architecture may determine that a biological condition is present with respect to a given individual.

In one or more additional examples, the one or more queries may be image-based and the computing architecture may analyze images included in the corpus of information with respect to template images. The template images may be generated based on analyzing a number of images in which a biological condition is present and aggregating the number of images into a template image. In these scenarios, the computing architecture may analyze images included in the corpus of information with respect to one or more template images to determine a measure of similarity between the images included in the corpus of information and the template images. In situations where the measure of similarity for an individual is at least a threshold value, the computing architecture may determine that a characteristic of a biological condition is present in the individual.

After determining individuals having one or more characteristics, the computing architecture may, at operation, generate data structures that store data for individuals having the one or more characteristics. In one or more examples, the computing architecture may generate data tables that indicate individuals having an individual characteristics and/or individuals having a group of characteristics. For example, the computing architecture may generate a first data table and a second data table. The first data table may indicate individuals having one or more first characteristics and the second data table may indicate individuals having one or more second characteristics. In one or more illustrative examples, the first data table may indicate individuals having one or more first biomarkers for a biological condition and the second data table may indicate individual having one or more second biomarkers for the biological condition. The one or more first biomarkers may correspond to one or more first genomic and/or epigenomic variants that are associated with the biological condition and the one or more second biomarkers may correspond to one or more second genomic and/or epigenomic variants that are associated with the biological condition.

One or more data structures may be generated from the corpus of information that store identifiers of the portion of the subset of the additional group of individuals and that store an indication that the portion of the subset of the additional group of individuals corresponds to the one or more biomarkers. The one or more data structures may be stored by an intermediate data repository. One or more de-identification operations may be performed with respect to the identifiers of the portion of the subset of the additional group of individuals before modifying the integrated data repository to store at least a portion of the additional information of the medical records of the portion of the subset of the additional group of individuals in relation to the number of identifiers. After de-identification of the information stored by the one or more data structures, the information stored by the integrated data repository may be added to the integrated data repository. In at least some examples, the de-identified medical records information may be added to the integrated data repository in addition to or in lieu of the health insurance claims data. In various examples, the one or more data structures storing the de-identified medical records information with respect to the biomarker data may have one or more logical connections with other data structures stored in the integrated data repository. To illustrate, the one or more data structures storing the de-identified medical records information with respect to the biomarker data may have one or more logical connections with at least one of the first data table may store information corresponding to a panel used to generate genomics data, mutations of genomic and/or epigenomic regions, types of mutations, copy numbers of genomic and/or epigenomic regions, coverage data indicating numbers of nucleic acid molecules identified in a sample having one or more mutations, testing dates, and patient information, the second data that stores data related to one or more patient visits by individuals to one or more healthcare providers, the a third data table that stores information corresponding to respective services provided to individuals with respect to one or more patient visits to one or more healthcare providers indicated by the second data table, the fourth data table that stores personal information of the group of individuals, the fifth data table that stores information related to a health insurance company or governmental entity that made payment for services provided to the group of individuals, the sixth data table storing information corresponding to health insurance coverage information for the group of individuals, such as a type of health insurance plan related to the group of individuals, or the seventh data table that stores information related to pharmaceutical treatments obtained by the group of individuals.

Described herein is a machine in the form of a computer system within which a set of instructions may be executed for causing the machine to perform any one or more of the methodologies discussed herein, according to an example, according to an example implementation. For example, a machine in the example form of a computer system, within which instructions (e.g., software, a program, an application, an applet, an app, or other executable code) for causing the machine to perform any one or more of the methodologies discussed herein may be executed. For example, the instructions may cause the machine to implement the architectures and frameworks described previously, and to execute the methods described with respect to previously. For example, one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described through instructions. For example, a machine can include a computing device with an analysis component. Analysis can include survival, modeling, sub-modeling, disease node determination and identification (e.g., driver mutations), disease association, disease subtyping, recurrence, metastasis, time to next treatment, etc. In various embodiments analysis components are embodied in machine-executable components within a system including various electronic data sources and data structures comprising information capable of use with the analysis component. Non-limiting examples include data sources and structures such survival information, genetic information, model data, sub-model, disease node determination and identification, disease association information, disease subtyping, recurrence, metastasis, time to next treatment, etc.

A computing device can include or be operatively coupled to at least one memory and at least one processor. The at least one memory stores executable instructions for performance of analysis when executed by the at least one processor. In some embodiments, the memory can also store the various data sources and/or structures of system. In other embodiments, the various data sources and structures of system can be stored in other memory (e.g., at a remote device or system), that is accessible to the computing device.

The instructions transform the general, non-programmed machine, such as a computing device, into a particular machine programmed to carry out the described and illustrated functions in the manner described. In alternative implementations, the machine operates as a standalone device or may be coupled (e.g., networked) to other machines. In a networked deployment, the machine may operate in the capacity of a server machine or a client machine in a server-client network environment, or as a peer machine in a peer-to-peer (or distributed) network environment. The machine may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions, sequentially or otherwise, that specify actions to be taken by the machine. One of skill appreciate that a machine include a collection of machines that individually or jointly execute the instructions to perform any one or more of the methodologies discussed herein.

Examples of computing devices may include logic, one or more components, circuits (e.g., modules), or mechanisms. Circuits are tangible entities configured to perform certain operations. In an example, circuits may be arranged (e.g., internally or with respect to external entities such as other circuits) in a specified manner. In an example, one or more computer systems (e.g., a standalone, client or server computer system) or one or more hardware processors (processors) may be configured by software (e.g., instructions, an application portion, or an application) as a circuit that operates to perform certain operations as described herein. In an example, the software may reside (1) on a non-transitory machine readable medium or (2) in a transmission signal. In an example, the software, when executed by the underlying hardware of the circuit, causes the circuit to perform the certain operations.

The various operations of method examples described herein may be performed, at least partially, by one or more processors that are temporarily configured (e.g., by software) or permanently configured to perform the relevant operations. Whether temporarily or permanently configured, such processors may constitute processor-implemented circuits that operate to perform one or more operations or functions. In an example, the circuits referred to herein may comprise processor-implemented circuits.

Similarly, the methods described herein may be at least partially processor implemented. For example, at least some or all of the operations of a method may be performed by one or processors or processor-implemented circuits. The performance of certain of the operations may be distributed among the one or more processors, not only residing within a single machine, but deployed across a number of machines. In an example, the processor or processors may be located in a single location (e.g., within a home environment, an office environment or as a server farm), while in other examples the processors may be distributed across a number of locations.

The one or more processors may also operate to support performance of the relevant operations in a “cloud computing” environment or as a “software as a service.”

(SaaS). For example, at least some of the operations may be performed by a group of computers (as examples of machines including processors), with these operations being accessible via a network (e.g., the Internet) and via one or more appropriate interfaces (e.g., Application Program Interfaces (APIs).)

Example implementations (e.g., apparatus, systems, or methods) may be implemented in digital electronic circuitry, in computer hardware, in firmware, in software, or in any combination thereof. Example implementations may be implemented using a computer program product (e.g., a computer program, tangibly embodied in an information carrier or in a machine readable medium, for execution by, or to control the operation of, data processing apparatus such as a programmable processor, a computer, or multiple computers).

A computer program may be written in any form of programming language, including compiled or interpreted languages, and it may be deployed in any form, including as a stand-alone program or as a software module, subroutine, or other unit suitable for use in a computing environment. A computer program may be deployed to be executed on one computer or on multiple computers at one site or distributed across multiple sites and interconnected by a communication network.

In an example, operations may be performed by one or more programmable processors executing a computer program to perform functions by operating on input data and generating output. Examples of method operations may also be performed by, and example apparatus may be implemented as, special purpose logic circuitry (e.g., a field programmable gate array (FPGA) or an application-specific integrated circuit (ASIC)).

The computing system may include clients and servers. A client and server are generally remote from each other and generally interact through a communication network. The relationship of client and server arises by virtue of computer programs running on the respective computers and having a client-server relationship with each other. In implementations deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., computing device) and software architectures that may be deployed in example implementations.

In an example, the computing device may operate as a standalone device or the computing device may be connected (e.g., networked) to other machines.

In a networked deployment, the computing device may operate in the capacity of either a server or a client machine in server-client network environments. In an example, computing device may act as a peer machine in peer-to-peer (or other distributed) network environments. The computing device may be a personal computer (PC), a tablet PC, a set-top box (STB), a mobile telephone, a web appliance, a network router, switch or bridge, or any machine capable of executing instructions (sequential or otherwise) specifying actions to be taken (e.g., performed) by the computing device. Further, while only a single computing device is illustrated, the term “computing device” shall also be taken to include any collection of machines that individually or jointly execute a set (or multiple sets) of instructions to perform any one or

The computing device may additionally include a storage device (e.g., drive unit), a signal generation device (e.g., a speaker), a network interface device, and one or more sensors, such as a global positioning system (GPS) sensor, compass, accelerometer, or another sensor. The storage device may include a machine readable medium on which is stored one or more sets of data structures or instructions (e.g., software) embodying or utilized by any one or more of the methodologies or functions described herein. The instructions may also reside, completely or at least partially, within the main memory, within static memory, or within the processor during execution thereof by the computing device. In an example, one or any combination of the processor, the main memory, the static memory, or the storage device may constitute machine readable media.

While the machine readable medium is illustrated as a single medium, the term “machine readable medium” may include a single medium or multiple media (e.g., a centralized or distributed database, and/or associated caches and servers) that configured to store the one or more instructions. The term “machine readable medium” may also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions.

As used herein, a component, may refer to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

### Diseases

The present methods can be used to diagnose presence of conditions, in a subject, to characterize conditions, monitor response to treatment of a condition, effect prognosis risk of developing a condition or subsequent course of a condition. The present disclosure can also be useful in determining the efficacy of a particular treatment option. Successful treatment options may increase the amount of nucleic acids, such as cell free nucleic acids, detected in subject's blood if the treatment is successful as diseased and dysfunctional die and shed DNA or otherwise exhibit chronic and acute signs of inflammation. In other examples, this may not occur. In another example, perhaps certain treatment options may be correlated with genetic profiles of disease types and sub-types over time. This correlation may be useful in selecting a therapy.

In some embodiments, the methods and systems disclosed herein may be used to identify customized or targeted therapies to treat a given disease or condition in patients based on the classification of a nucleic acid variant as being of somatic or germline origin. Typically, the disease under consideration is a type of cancer.

Further, the methods of the disclosure may be used to characterize the heterogeneity of an abnormal condition in a subject. Such methods can include, e.g., generating a genomic and epigenomic profile of extracellular polynucleotides derived from the subject, wherein the genetic profile includes a plurality of data that can characterize malfunctions and abnormalities associated with the heart muscle and valve tissues (e.g., hypertrophy), the decreased supply of blood flow and oxygen supply to the heart are often secondary symptoms of debilitation and/or deterioration of the blood now and supply system caused by physical and biochemical stresses. Examples of cardiovascular diseases that are directly affected by these types of stresses include atherosclerosis, coronary artery disease, peripheral vascular disease and peripheral artery disease, along with various cardias and arrythmias which may represent other forms of disease and dysfunction. The present methods can be used to generate our profile, fingerprint or set of data that is a summation of genetic information derived from different cells in a heterogeneous disease. This set of data may comprise copy number variation, epigenetic variation, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

### Therapies and Related Administration

In certain embodiments, the methods disclosed herein relate to identifying and administering customized therapies to patients given the status of a nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the test subject and/or patients who are receiving, or who have received, the same therapy as the test subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraarticular, which administration may include tablets, capsules, granules, aqueous suspensions, gels, sprays, suppositories, salves, ointments, or the like.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it should be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

### Biomarkers

The disclosure provides methods of using biomarkers for the diagnosis, prognosis, and therapy selection of a subject suffering from diseases, e.g., heart failure, cardiovascular disease, cancer, etc. A biomarker may be any gene or variant of a gene whose presence, mutation, deletion, substitution, copy number, or translation (i.e., to a protein) is an indicator of a disease state. Biomarkers of the present disclosure may include the presence, mutation, deletion, substitution, copy number, or translation in any one or more of EGFR, KRAS, MET, BRAF, MYC, NRAS, ERBB2, ALK, Notch, PIK3CA, APC, and SMO.

A biomarker is a genetic variant. Biomarkers may be determined using any of several resources or methods. A biomarker may have been previously discovered or may be discovered de novo using experimental or epidemiological techniques. Detection of a biomarker may be indicative of a disease when the biomarker is highly correlated to the disease. Detection of a biomarker may be indicative of cancer when a biomarker in a region or gene occur with a frequency that is greater than a frequency for a given background population or dataset.

Publicly available resources such as scientific literature and databases may describe in detail genetic variants. Scientific literature may describe experiments or genome-wide association studies (GWAS) associating one or more genetic variants. Databases may aggregate information gleaned from sources such as scientific literature to provide a more comprehensive resource for determining one or more biomarkers. Non-limiting examples of databases include FANTOM, GTex, GEO, Body Atlas, INSiGHT, OMIM (Online Mendelian Inheritance in Man, omim.org), cBioPortal (cbioportal.org), CIViC (Clinical Interpretations of Variants in Cancer, civic.genome.wustl.edu), DOCM (Database of Curated Mutations, docm.genome.wustl.edu), and ICGC Data Portal (dcc.icgc.org). In a further example, the COSMIC (Catalogue of Somatic Mutations in Cancer) database allows for searching of biomarkers by cancer, gene, or mutation type. Biomarkers may also be determined de novo by conducting experiments such as case control or association (e.g, genome-wide association studies) studies.

One or more biomarkers may be detected in the sequencing panel. A biomarker may be one or more genetic variants. Biomarkers can be selected from single nucleotide variants (SNVs), copy number variants (CNVs), insertions or deletions (e.g., indels), gene fusions and inversions. Biomarkers may affect the level of a protein. Biomarkers may be in a promoter or enhancer, and may alter the transcription of a gene. The biomarkers may affect the transcription and/or translation efficacy of a gene. The biomarkers may affect the stability of a transcribed mRNA. The biomarker may result in a change to the amino acid sequence of a translated protein. The biomarker may affect splicing, may change the amino acid coded by a particular codon, may result in a frameshift, or may result in a premature stop codon. The biomarker may result in a conservative substitution of an amino acid. One or more biomarkers may result in a conservative substitution of an amino acid. One or more biomarkers may result in a nonconservative substitution of an amino acid.

The frequency of a biomarker may be as low as 0.001%. The frequency of a biomarker may be as low as 0.005%. The frequency of a biomarker may be as low as 0.01%. The frequency of a biomarker may be as low as 0.02%. The frequency of a biomarker may be as low as 0.03%. The frequency of a biomarker may be as low as 0.05%. The frequency of a biomarker may be as low as 0.1%. The frequency of a biomarker may be as low as 1%.

No single biomarker may be present in more than 50%, of subjects having the cancer. No single biomarker may be present in more than 40%, of subjects having the cancer. No single biomarker may be present in more than 30%, of subjects having the cancer. No single biomarker may be present in more than 20%, of subjects having the cancer. No single biomarker may be present in more than 10%, of subjects having the cancer. No single biomarker may be present in more than 5%, of subjects having the cancer. A single biomarker may be present in 0.001% to 50% of subjects having cancer. A single biomarker may be present in 0.01% to 50% of subjects having cancer. A single biomarker may be present in 0.01% to 30% of subjects having cancer. A single biomarker may be present in 0.01% to 20% of subjects having cancer. A single biomarker may be present in 0.01% to 10% of subjects having cancer. A single biomarker may be present in 0.1% to 10% of subjects having cancer. A single biomarker may be present in 0.1% to 5% of subjects having cancer.

### Genetic Analysis

Genetic analysis includes detection of nucleotide sequence variants and copy number variations. Genetic variants can be determined by sequencing. The sequencing method can be massively parallel sequencing, that is, simultaneously (or in rapid succession) sequencing any of at least 100,000, 1 million, 10 million, 100 million, or 1 billion polynucleotide molecules. Sequencing methods may include, but are not limited to: high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next-generation sequencing, Single Molecule Sequencing by Synthesis (SMSS) (Helicos), massively-parallel sequencing, Clonal Single Molecule Array (Solexa), shotgun sequencing, Maxam-Gilbert or Sanger sequencing, primer walking, sequencing using PacBio, SOLiD, Ion Torrent, or Nanopore platforms and any other sequencing methods known in the art.

Sequencing can be made more efficient by performing sequence capture, that is, the enrichment of a sample for target sequences of interest, e.g., sequences including the KRAS and/or EGFR genes or portions of them containing sequence variant biomarkers. Sequence capture can be performed using immobilized probes that hybridize to the targets of interest.

Cell free DNA can include small amounts of tumor DNA mixed with germline DNA. Sequencing methods that increase sensitivity and specificity of detecting tumor DNA, and, in particular, genetic sequence variants and copy number variation, can be useful in the methods of this invention. Such methods are described in, for example, in WO 2014/039556. These methods not only can detect molecules with a sensitivity of up to or greater than 0.1%, but also can distinguish these signals from noise typical in current sequencing methods. Increases in sensitivity and specificity from blood-based samples of cfDNA can be achieved using various methods. One method includes high efficiency tagging of DNA molecules in the sample, e.g., tagging at least any of 50%, 75% or 90% of the polynucleotides in a sample. This increases the likelihood that a low-abundance target molecule in a sample will be tagged and subsequently sequenced, and significantly increases sensitivity of detection of target molecules.

Another method involves molecular tracking, which identifies sequence reads that have been redundantly generated from an original parent molecule, and assigns the most likely identity of a base at each locus or position in the parent molecule. This significantly increases specificity of detection by reducing noise generated by amplification and sequencing errors, which reduces frequency of false positives.

Methods of the present disclosure can be used to detect genetic variation in non-uniquely tagged initial starting genetic material (e.g., rare DNA) at a concentration that is less than 5%, 1%, 0.5%, 0.1%, 0.05%, or 0.01%, at a specificity of at least 99%, 99.9%, 99.99%, 99.999%, 99.9999%, or 99.99999%. Sequence reads of tagged polynucleotides can be subsequently tracked to generate consensus sequences for polynucleotides with an error rate of no more than 2%, 1%, 0.1%, or 0.01%.

In other examples, a gene of interest may be amplified using primers that recognize the gene of interest. The primers may hybridize to a gene upstream and/or downstream of a particular region of interest (e.g., upstream of a mutation site). A detection probe may be hybridized to the amplification product. Detection probes may specifically hybridize to a wild-type sequence or to a mutated/variant sequence. Detection probes may be labeled with a detectable label (e.g., with a fluorophore). Detection of a wild-type or mutant sequence may be performed by detecting the detectable label (e.g., fluorescence imaging). In examples of copy number variation, a gene of interest may be compared with a reference gene. Differences in copy number between the gene of interest and the reference gene may indicate amplification or deletion/truncation of a gene. Examples of platforms suitable to perform the methods described herein include digital PCR platforms such as e.g., Fluidigm Digital Array.

Described herein is a method for analysis of nucleic acid sequence information. In various embodiments, the method of analysis comprises one or more models, each of one or more including one or more of survival, sub-modeling, disease node determination and identification (e.g., driver mutations), disease association, disease subtyping, recurrence, metastasis, time to next treatment, etc. as separate components. In various embodiments, a model includes hierarchal models (e.g., nested models, multi-level models), mixed models (e.g., regression such as logistic regression and Poisson regression, pooled, random effect, fixed effect, mixed effect, linear mixed effect, generalized linear mixed effect), hazard model, odds ratio models and/or repeated sample (e.g., repeated measures such as ANOVA). In various embodiments, the model is a hierarchical random effects model. In various embodiments, the model is a hierarchical cubic spline random effects model. In various embodiments, the model is a cubic spline model. In various embodiments, the model is a generalized linear effects model. In various embodiments, the model is a linear effects model. In various embodiments, the model is a Cox proportional hazard model. In various embodiments, the method of analysis comprises assembly of models together. In various embodiments assembly includes generation of association parameters. In one or more embodiments, the method of analysis includes patient survival information and patient genetic information. As an example, assembly of models together could include different models for the different types of cancers, including subtypes, represented in the patient survival information. Each of different models can be configured to determine correlations between genetic factors and the survival times of patients diagnosed with the respective types of cancers they are configured to evaluate. For example, genetic factors determined to have strong correlations to cancer survival times (e.g., relatively short survival times and/or relatively long survival times) can be recommended as potential therapeutic targets.

In various embodiments, analysis can include one or more of survival, sub-modeling, disease node determination and identification (e.g., driver mutations), disease association, disease subtyping, recurrence, metastasis, time to next treatment, etc. as separate components. For example modeling can facilitate applying the aforementioned, such as patient survival information and the patient genetic information. In various embodiments, a sub-modeling component can determine subsets of the patient survival information and the patient genetic information for generations different patient cohorts associated with different types of cancer and cancer subtypes. In various embodiments, a sub-model includes hierarchal models (e.g., nested models, multi-level models), mixed models (e.g., regression such as logistic regression and Poisson regression, pooled, random effect, fixed effect, mixed effect, linear mixed effect, generalized linear mixed effect), hazard model, odds ratio models and/or repeated sample (e.g., repeated measures such as ANOVA). In various embodiments, the sub-model is a hierarchical random effects model. In various embodiments, the sub-model is a hierarchical cubic spline random effects model. In various embodiments, the sub-model is a cubic spline model. In various embodiments, the sub-model is a generalized linear effects model. In various embodiments, the sub-model is a linear effects model. In various embodiments, the sub-model is a Cox proportional hazard model. Each subset of the patient survival information and the patient genetic information can comprise information for patients diagnosed with a different type of cancer and cancer subtypes. For example, the sub-modeling component can further apply the subsets of the patient survival information and the patient genetic information to corresponding individual survival models developed for the different cancer types, including subtypes. In various embodiments, information generated for the method of analysis can be stored in memory (e.g., as model data). In various embodiments, and information generated for the method of analysis generates one or more survival models for individual subjects.

In various embodiments, analysis of the patient survival information and the patient genetic information using the survival models, include the disease node determination and identification component can identify, for each type of cancer, disease nodes included in the patient genetic information that are involved in the genetic mechanisms employed by the respective cancer types to proliferate. In various embodiments, the disease node component identifies disease node based on observed correlations between genetic factors and the cancer survival times provided in the patient survival information. For example, a genetic factor that is frequently observed in association with short survival times of a specific type of cancer and less frequently observed in association with long survival times of the specific type of cancer can be identified as an active genetic factor having an active role in the genetic mechanism of the specific type of cancer, including subtypes.

In various embodiments, disease node determination and identification includes disease association parameters regarding associations between different cancer types to facilitate identifying the active genetic factors associated with the different cancer types. For example, cancer types which are highly associated can share one or more common critical underlying genetic factors. As readily appreciated by one of ordinary skill, models (e.g., survival model) of associated cancer types dialectically exchange information to determine and/or identify active genetic factors across types of cancer, including subtypes. In various embodiments, the disease association parameters applied by the disease node determination and identification is facilitated by modeling. In various embodiments, generation of individual survival models can employ one or more machine learning algorithms to facilitate the determination and/or identification of the survival, modeling, disease node associated with a particular type of cancer, including subtypes, based on the, and the patient genetic information and the disease association parameters.

In some embodiments, in association with node determination and identification for cancer type, including subtypes, includes determination of a score system for the disease node(s). For example, a score for a disease node with respect to a specific type of cancer, including subtypes, reflects association of the disease node to the survival time of the specific type of cancer, including subtypes. In various embodiments, scores can be based on a frequency with which a particular genetic factor is directly or indirectly identified for patients diagnosed with a specific cancer type. In various embodiments, analysis includes the aforementioned survival, sub-modeling, disease node determination and identification (e.g., driver mutations), disease association, disease subtyping, recurrence, metastasis, time to next treatment, etc. can be related to, less than a defined threshold, greater than a defined threshold. For example, greater scores associated with a disease node and cancer type, including, the greater the contribution of the disease node to survival time. In various embodiments, formation regarding disease nodes for respective types of cancer, including subtypes and scores determined for the active genetic factors can be collated in a data structure, such as a database.

Described herein is a method of analysis that includes effects modeling. In various embodiments, the effects modeling includes random effect, fixed effect, mixed effect, linear mixed effect, and generalized linear mixed effect. In various embodiments, the effects includes cubic spline. In various embodiments, the effects modeling includes regression. In various embodiments, the effects modeling includes logistic and Poisson regression. In various embodiments, the model does not include covariates. In In various embodiments, the model includes covariates. In various embodiments, the covariates are information from medical records (including laboratory testing records such as genomic, epigenomic, nucleic acid and other analyte results), insurance records or the like. Examples include age, line of therapies, smoking status (yes/no), gender, and various scoring and/or staging systems that have been utilized for specific cancer disease patients, with an illustrative example including age (in years), line of anti-EGFR therapy, smoking status (yes/no), gender (female/male), and the Van Walraven Elixhauser Comorbidity (ELIX) score specific to lung cancer patients (expressed as a weighted measure across multiple common comorbidities. One of skill readily appreciates covariates can include any number of data elements for individuals and individuals in a population, such as that from medical records (including laboratory testing records such as genomic, epigenomic, nucleic acid and other analyte results), insurance records or the like.

In various embodiments, the method of analysis includes generation of a hierarchy including at least one first level equation. In various embodiments, a first level equation includes a truncated cubic spline. In various embodiments, the truncated cubic spline includes longitudinal data. This includes, for example, direct or indirect measurements of ctDNA levels, allele fractions, tumor fractions. In various embodiments, an additional level equation includes covariate. In various embodiments, the covariates are information for an individual, or individuals in a population, drawn and/or stored from medical records (including laboratory testing records such as genomic, epigenomic, nucleic acid and other analyte results), insurance records, or the like. Examples include age, line of therapies, smoking status (yes/no), gender, and various scoring and/or staging systems that have been utilized for specific cancer disease patients. In various embodiments, a velocity plot is generated. In various embodiments, the velocity plot is a derivative or one or equations, such as at least one first level equation. In various embodiments, the method of analysis includes one or more of Equations (1), (2) and (3) described in the Examples.

Described herein is a method of analysis that includes jointly solving different analysis components, including one or more of survival, modeling and sub-modeling, disease node determination and identification (e.g., driver mutations), disease association, disease subtyping, recurrence, metastasis, time to next treatment, etc. as separate components. In various embodiments, the method of analysis includes jointly solving one or more different models for the different cancer types under a joint model framework. For example, the method of analysis could include jointly solving one or more different survival models for the different cancer types under a joint model framework. In various embodiments, the method includes determination of association parameters. In various embodiments, association parameters include, for example, the relationship between patient survival and the patient's estimated current value of the biomarker, the relationship between patient survival and patient's estimated current change over time with respect to the biomarker. In various embodiments, this includes the slope, and the relationship between overall survival and current estimated area under a subject's longitudinal trajectory as a surrogate for a biomarker's cumulative effect. It is readily appreciated by one of ordinary skill that association parameters can undertake multiple forms, and can also be combined. For instance, one could examine the relationship between overall survival and estimated current value plus the estimated current slope of the patient's longitudinal trajectory.

### Example 1—Joint Modeling

The Inventors applied joint modeling of longitudinal and time-to-event data (JM) combined with next generation sequencing (NGS) genetic testing to demonstrate the capability of detecting change in a biomarker (or several biomarkers) over time as related to a specific patient's probability of survival. Detecting and characterization of genomic biomarkers with the methods and techniques illustrates how the evolution of such biomarkers can be associated with, and predictive of, patient survival. This real world applications of joint modeling, as one example, generates patient-level monitoring systems designed to enhance the decision making capabilities of clinicians.

Notably, JMs include the ability to properly accommodate endogenous time varying covariates. As most biomarkers fall into this category, this leads to a reduction in the bias of parameter estimates, improved statistical inference, and the ability to make dynamic patient-level predictions where predictions are based on portions of, or the complete biomarker history. Joint modeling is flexible in that both frequentist and Bayesian approaches have been developed. Here, the Inventors adopted a Bayesian approach based on a Markov Chain Monte Carlo sampling algorithm for computational efficiency.

### Example 2—Genetic Testing Via Next Generation Sequencing

The Inventors selected a cohort of patients from a real world evidence database which includes real-world outcomes, anonymized genomic data, and structured payer claims data for >240,000 patients. For demonstrative purposes, different target populations within this dataset included patients diagnosed with non-small cell lung cancer (NSCLC) that possess an EGFR L858R mutation, colorectal cancer (CRC) with KRAS G12D and KRAS12V, each separately. Due to the longitudinal component of this study only patents with a minimum of three temporal measures were included. Upon satisfying these conditions, the resulting cohort consisted of 252 patients. The biomarkers of interest i.e. the longitudinal outcomes are the patient's mutant allele frequency (AF) and tumor fraction (TF), where it is the progression of these biomarkers over time that we intended to associate with patient survival.

### Examples 3—Methods

Joint modeling framework is divided into evaluating two sub-models, where, once sub-models are analyzed, information from these sub-models are combined with the purpose of determining if an association between the two exists. More specifically, the first sub-model focuses on providing a sufficient representation of the longitudinal data (at the patient-level), and the second sub-model assesses patient survival. In this study a general linear mixed model (GLMM) was employed to assess the temporal progression of each biomarker while a Cox-proportional hazard (CPH) model examined patient survival. It is important to note that since the distribution of each biomarker is highly skewed, to align with GLMM normality assumptions, analysis was based on the log transformation of both AF and TP. Furthermore, due to several patients exhibiting complex biomarker progressions, a cubic spline model was used to describe patient level response. Moreover, since factors such as age and gender are often confounded with survival, these factors were included in the CPH model to act as statistical controls.

As readily appreciated by one of ordinary skill, the methods and techniques described herein support determination of associations between the longitudinal and time-to-event data referred to as the association structure. Examples of association structure includes, but is not limited to, the relationship between patient survival and the patient's estimated current value of the biomarker, the relationship between patient survival and patient's estimated current change over time with respect to the biomarker e.g., the slope, and the relationship between overall survival and current estimated area under the patient's longitudinal trajectory, which, is often used as surrogate for the biomarker's cumulative effect. Associate structures can undertake multiple forms, and can also be combined. For instance, one could examine the relationship between overall survival and estimated current value plus the estimated current slope of the patient's longitudinal trajectory. Here, the Inventors describe an association structure to the current value, slope, and their combination, though, one of skill in the art will appreciate that a multitude of association structures—many of which are not explicitly mentioned above but readily known by one of skill in the art—are available for exploration. After establishing a suitable JM for each biomarker, these JMs will in turn be used to inform dynamic predictions. That is, overall survival is predicted for each patent depending upon the nature of the association structure between the longitudinal and time-to-event data, or, more specifically, survival is predicted for a given patient using measures captured up to a given time point, and, as additional measures are collected, patient survival predictions adjust accordingly—hence the term “dynamic predictions”.

### Example 4—Statistical Analysis

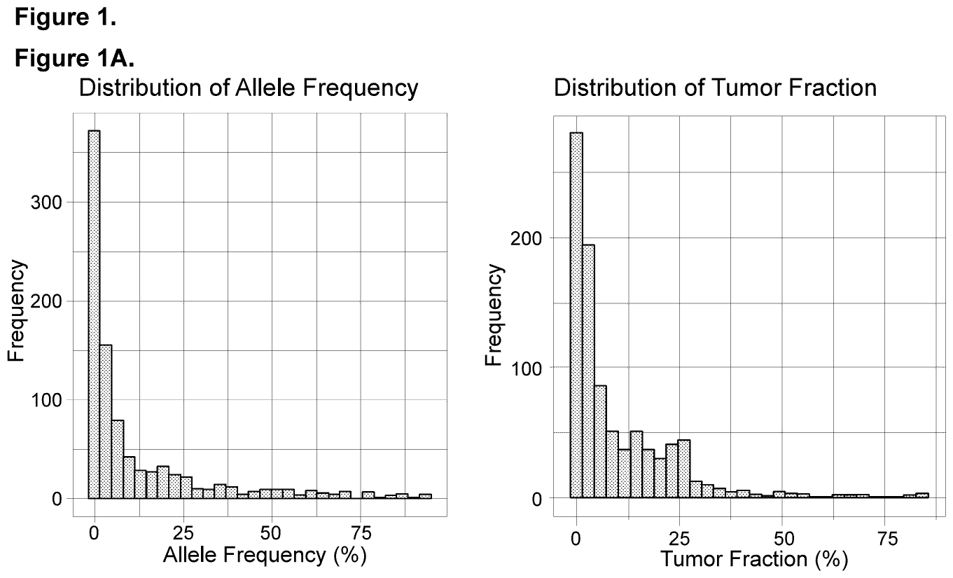

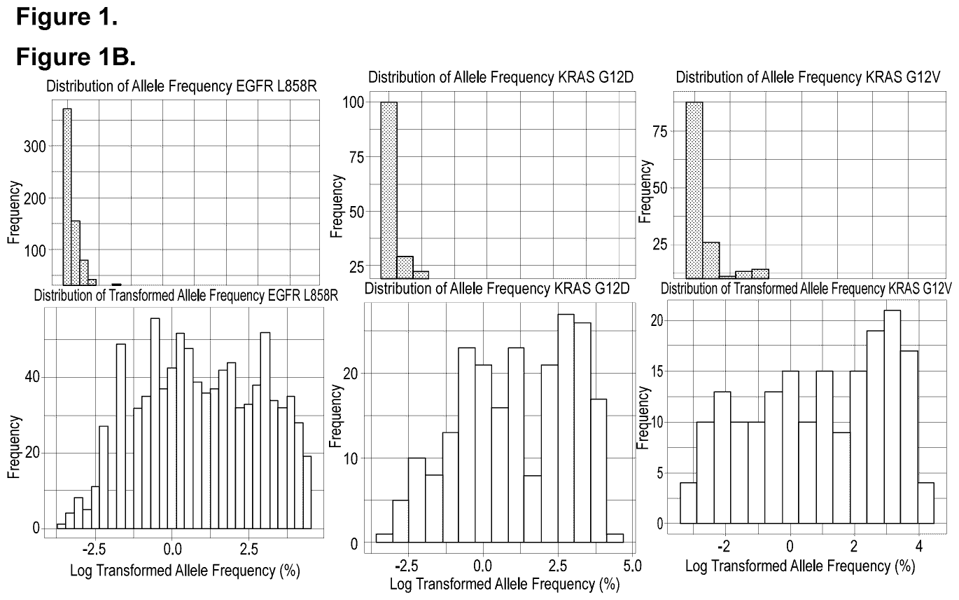

All statistical analysis was performed using R version 4.1.3 where the JMBayes2 package executed the joint modeling. As previously mentioned, due to the study's longitudinal component, each patent possessed a minimum of three temporal measures, where the first measure coincides with the patient's initial Guardant360 test while remining measures follow suit accordingly. In all, 252 patients met these criteria resulting in a total of 909 measures collected on AF and TP respectively where measures spanned from Nov. 19, 2014 to Sep. 30, 2022. The distribution of each biomarker is given in FIG. 1 followed by the associated summary statistics (see Table 1). JM results indicate that the most recent change over time of each biomarker is associated with patient survival (AF: p-value=0.0139; TF: p-value=0.0332). Through these associations, graphical renderings of patient-level survival curves can be displayed to assess clinical outcome based on the patient's unique biomarker evolution.

TABLE 1
Summary Statistics for Allele Frequency and Tumor Fraction

| | | | | | | | Stand. |
|---|---|---|---|---|---|---|---|
| Biomarker | Min. | 1Q | Median | Mean | 3Q | Maximum | Dev. |
| Allele | 0.03 | 0.60 | 2.90 | 11.88 | 15.50 | 93.20 | 18.88 |
| Frequency | | | | | | | |
| Tumor | 0.04 | 1.00 | 4.00 | 9.44 | 14.80 | 84.10 | 12.28 |
| Fraction | | | | | | | |

### Example 5—Results

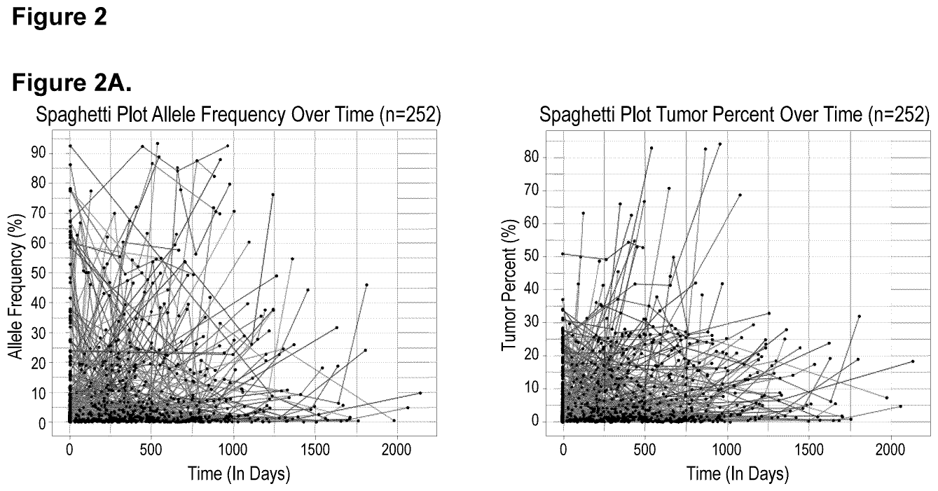

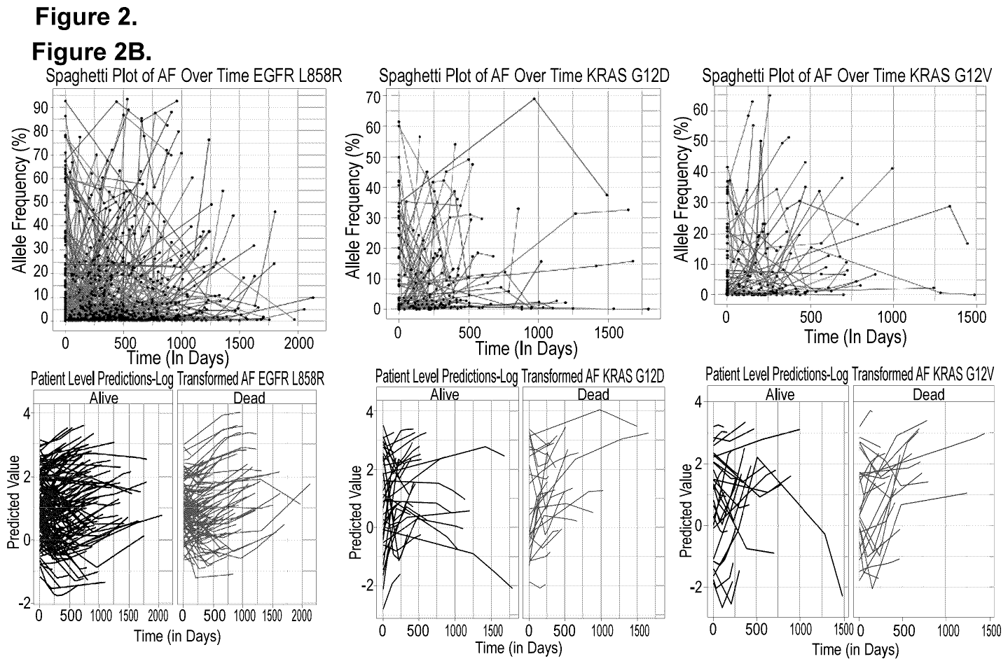

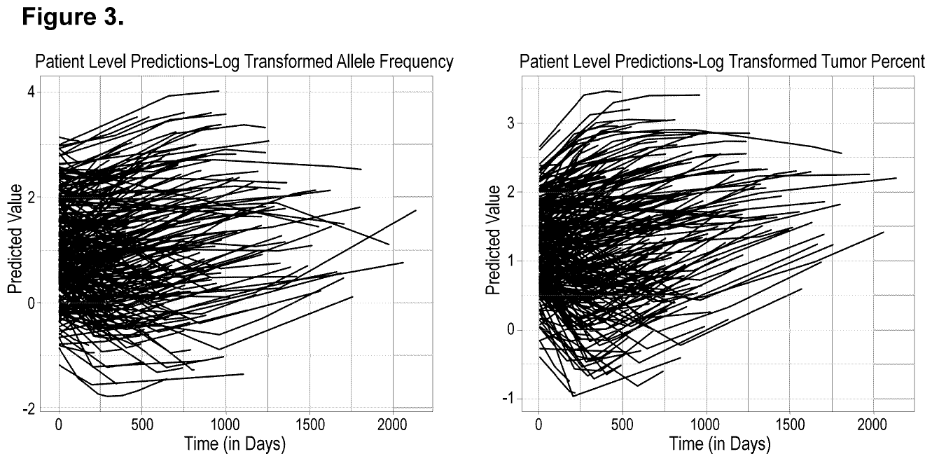

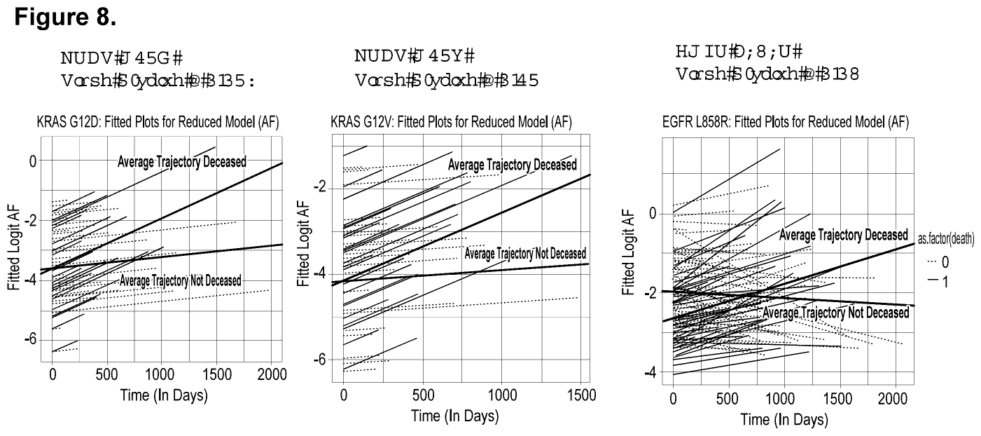

Distribution of Allele Frequency and Tumor Fraction is shown in FIG. 1. To complement the descriptive statistics, the patent-level longitudinal data for each biomarker is displayed as spaghetti plots in FIG. 2, which illustrates the complexity of the patient-level longitudinal progression for each biomarker and reinforces the skewed nature of the data. To adhere to the normality assumption required by the GLMM, a log transformation was applied to each biomarker, and, to compensate for the complexity observed in the patient level evolutions, a natural cubic spline was leveraged to model the longitudinal characteristics of each patient within the GLMM structure. Plots of fitted GLMM results for each patient are found in FIG. 3 and the fixed and random effects of the GLMM for each biomarker are depicted in FIG. 8. As biomarkers are collected on the same set of patients, only a single CPH model needs to be fit. Of the 252 patients, 99 experienced the event (death) whereas the remaining observations were censored. Since both age and gender are often confounded with survival, the initial CPH model included these covariates as statistical controls. However, analysis of the initial model revealed that neither age (p-value=0.519) nor gender (p-value 0.310) was statistically significant at the 0.05 level. Similarly, models that included age and gender separately produced similar results (age, p-value=0.56) and (gender, p-value=0.33). Subsequently, a null CPH model (a model absent of covariates) was employed for the joint modeling.

### Example 6—Fitting

Fitted Cubic Spline Based GLMM Results for Log Transformed Biomarkers is shown in FIG. 3. Three JMs were analyzed for each biomarker, each matching the association structures (and combinations thereof) cited earlier. Since the analysis was performed under a Bayesian paradigm, care was taken to ensure model parameters were estimated accurately. In doing so, each model consisted of two chains, where each chain had 9000 burn-in iterations followed by 90,000 iterations, and, to account for potential autocorrelation issues, a thinning factor of 3 was implemented. Likewise, inspection of trace plots provided visual conformation that model parameters converged adequately. Joint modeling results for each respective biomarker (numbered 1-3) are summarized in Tables 2 and 3. Since a Bayesian approach was adopted, 95% credible intervals, not frequentist-based confidence intervals are reported.

Results in Tables 2 and 3 reveal the second JM for each biomarker shows promise as evidenced by the respective p-values (0.0139 and 0.0332) suggesting an association between the current slope and patient survival exists. More information can be abstracted from these tables as well. That is, it is possible to calculate hazard ratios that correspond their respective association structures. For instance, referencing the mean in Table 2, if the current rate of change in allele frequency increases by 10% over 100 days, the resulting hazard ratio is 1.19, meaning the hazard of death related to such an increase is 19% greater. A similar calculation can be performed for the maximum tumor percent.

TABLE 2
Joint Modeling Results for the Log Transformation of Allele Frequency

| Model Number, | Association | | | | | |
|---|---|---|---|---|---|---|
| (DIC) | Structure | Mean | SD | 2.5% | 97.5% | p-value |
| 1, (5054.41) | Value | 0.047 | 0.126 | −0.1972 | 0.2935 | 0.7190 |
| 2, (5286.91) | Slope | 179.605 | 68.158 | 46.02 | 318.64 | 0.0139 |
| 3, (5375.00) | Value | −0.0915 | 0.1819 | −0.4775 | 0.2462 | 0.6260 |
| | Slope | 224.429 | 73.519 | 88.897 | 371.541 | 0.0059 |

1. Examination of density plots showed that, even though convergence was demonstrated, many posterior distributions were skewed, meaning that the deviance information criteria (DIC) may not be suitable for making model comparison as the distribution of the combined density plots is not multivariate normal. However, since it is often reported, the DIC is included above.
2. SD stands for Standard Deviation.

TABLE 3
Joint Modeling Results for the Log Transformation of Tumor Percent

| Model Number, | Association | | | | | |
|---|---|---|---|---|---|---|
| (DIC) | Structure | Mean | SD | 2.5% | 97.5% | p-value |
| 1, (4684.88) | Value | 0.0575 | 0.1616 | −0.2549 | 0.3776 | 0.7236 |
| 2, (4807.88) | Slope | 432.18 | 238.66 | 27.9398 | 971.68 | 0.0332 |
| 3, (4913.58) | Value | 0.3512 | 0.2434 | −0.0722 | 0.8848 | 0.1196 |
| | Slope | 627.54 | 232.41 | 217.52 | 1131.47 | 0.0010 |

1. Examination of density plots showed that, even though convergence was demonstrated, many posterior distributions were skewed, meaning that the deviance information criteria (DIC) may not be suitable for making model comparison as the distribution of the combined density plots is not multivariate normal. However, since it is often reported, the DIC is included above.
2. SD stands for Standard Deviation.

### Example 7—Dynamic Predictions

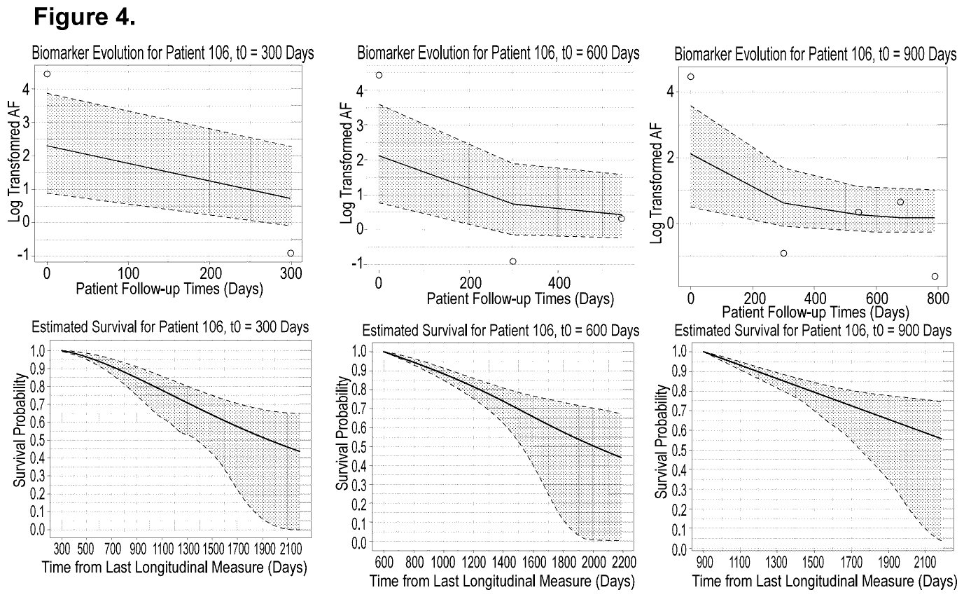

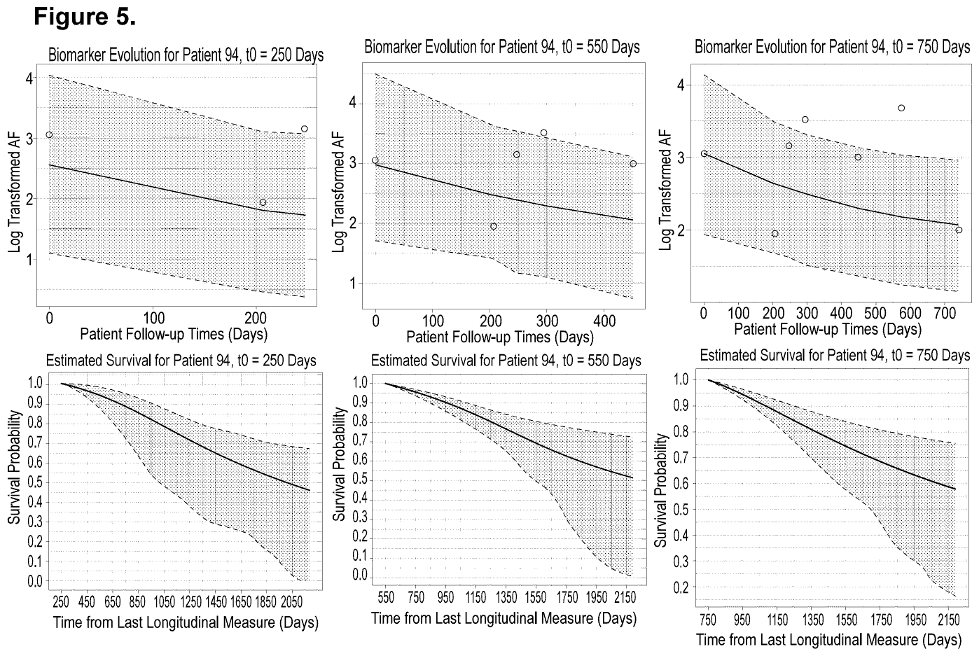

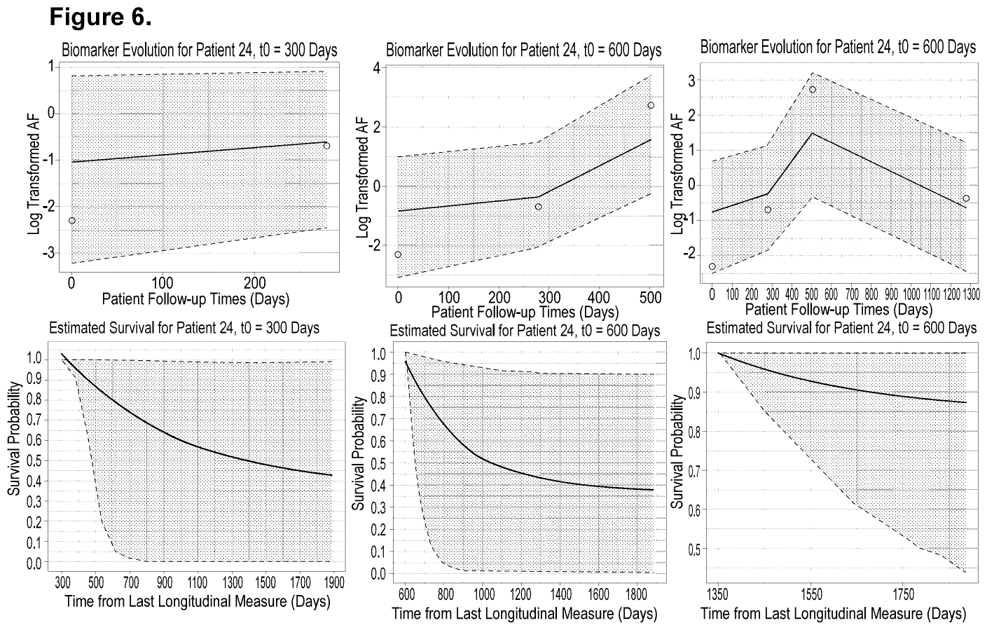

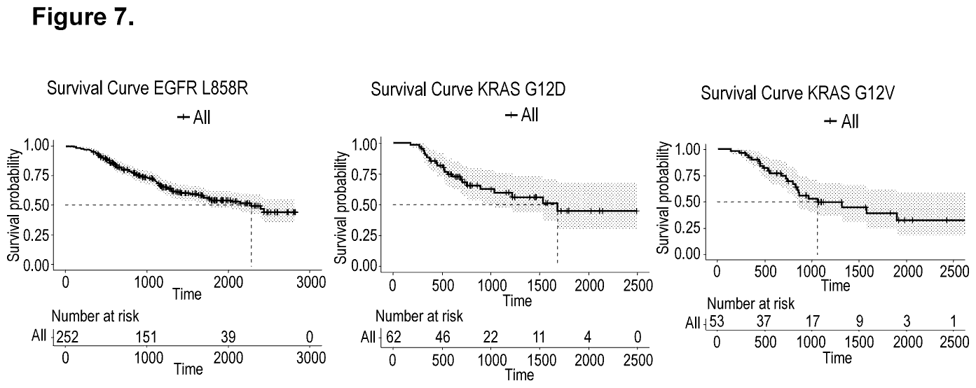

The HR does well to reflect an overall trend, but, from a precision medicine perspective, the real strength in the JM methodology lies in producing dynamic predictions. As the concept of dynamic prediction is best understood by visual representation, a graphical depiction of this process is provided in FIGS. 4 and 5.

The top panels in FIG. 4 portray the longitudinal trajectory (as seen from the blue lines) as related to the patient's biomarker evolution, where, as additional measures are captured, the trajectory adjusts accordingly. It is important to be mindful that focus is on the current slope of the trajectory as it is this association structure upon which the JM used in creating the dynamic predictions was built. In this example we examine timeframes that span from 0 to 300, 600, and 900 days respectively. Located directly below each trajectory i.e., the bottom panels are the matching survival curves. Notice that each curve is updated as new biomarker information becomes available. For instance, from 0 to 300 days the trajectory for patent 106 is decreasing as indicated by FIG. 4. Through examination of the corresponding survival curve, if we project out say 1000 days i.e., survival is evaluated at 1300 days, the patient's probability of survival is about 0.71 or 71%. Similarly, at 600 days, additional biomarker values are captured which alters the trajectory, where even though the trend remains downward the slope is now increasing. Evaluated at 1000 days out (at 1600 days) we see the patient's projected survival drops by 6%, from 71% to 65%. Such a result is expected, since, in general, as the slope increases, survival decreases. Finally, as the last set of measures collected up to 800 days cause a slight increase in the slope, the patient's estimated survival decreases marginally, from 65% to 64%. Here a projection of 1000 days was used; however, the survival trend remains relatively comparable regardless of the projected timeframe.

In contrast to patient 106, the slope of the trajectory for patient 94 (see FIG. 5) remains fairly consistent over the timespans considered, although a slight increase in the slopes is observed. As such we should expect to see little in the way of change in survival probabilities. If we project out 1000 days as before, the expected survival probabilities are 71%, 70%, and 69% respectively—which—align with expectations. As with the HR calculation, similar dynamic predictions can be made based on maximum tumor percent.

Using the methods and techniques described herein, JM results indicate that the most recent change over time of each biomarker is associated with patient survival (AF: p-value=0.0139; TF: p-value=0.0332). Through these associations, graphical renderings of patient-level survival curves can be displayed to assess clinical outcome based on the patient's unique biomarker evolution.

### Example 8—Discussion

In addition to the many available JM options, the dynamic prediction capabilities are particularly beneficial as they are well suited to enhance the decision-making capabilities of clinicians. This is because, in authentic medical environments, patient conditions are ever changing, and, consequently, it is often in the patient's best interest to make informed decisions using the most recent data available. As demonstrated, JMs intrinsically capture an ever-changing patient landscape, and, as shifts occur, the JM adapts accordingly. Therefore, by capitalizing on the ability of a JM to link up-to-date information to patient survival, clinicians can be well positioned to modify and/or adjust treatment plans with the ultimate goal of improving patient survival. Furthermore, the application of approaches such as JM supports generation of vast amounts of genetic data. One of skill in the art will appreciate that there are numerous biomarkers, cancer types, and mutations available for investigation as the analysis performed here can be applied to other cancer types and mutations, and, in the process, additional relevant biomarkers may be identified. This approach support creation of patient-specific monitoring systems that are both custom-tailored to a specific cancer type and mutation combination.

### Example 9—Hierarchical Cubic Spline Random Effects Model

Described herein is the use of hierarchical cubic spline random effects model (HCSREM) as applied to a retrospective real-world cohort of patients who were diagnosed with advanced non-small cell lung cancer (NSCLC). Here, of interest is ctDNA level as measured by the maximum variant allele fraction of all somatic variants detected through liquid biopsy, although one of ordinary skill appreciate although the proposed framework can be applied to an assortment of longitudinal biomarkers, the biomarker. A major advantage of the method is the ability to incorporate patient information, several relevant covariates were considered. Finally, to enhance interpretation, model results are displayed graphically in the form of estimated longitudinal projections, each based upon a patient's set of distinct traits. Within this process, patient-level projections are directly compared where comparisons are enhanced by velocity plots which are defined subsequently.

### Example 10—Data Source and Patient Cohort

The cohort used to illustrate the utility of the methodology is based on observational data and was sourced from the real world evidence anonymized clinical-genomic database, which includes structured commercial payer claims collected from inpatient and outpatient facilities in both academic and community settings.

Patients selected for the cohort were diagnosed with advanced non-small cell lung cancer (NSCLC) and had at least three genomic liquid biopsy tests in the US between Jun. 1, 2014 and Jun. 30, 2023. Only patients receiving targeted therapies for EGFR mutations were included, with the following therapies considered: osimertinib, afatinib, dacomitinib, erlotinib, gefitinib, and amivantamab. All patients were required to retain at least three blood samples while on a specific line of anti-EGFR therapy, or within 30 days prior to line of therapy initiation and 30 days post end line of therapy. Patients whose first genomic test on the line of therapy was more than 120 days after the start of line of therapy were excluded. For patients with multiple lines of therapy meeting these criteria, the earliest line of therapy was selected for study inclusion. Finally, patients with suspected germline mutations were removed from the cohort.

### Example 11—Response Variable and Study Covariates

The response variable, ctDNA measurements captured over time, is reported as a percentage. In instances where samples contained ctDNA levels below the assay's limit of detection, values were replaced with ctDNA levels of 0.04%, the lowest value in the cohort and consistent with the limit of detection of the test. All covariates except mortality were captured at baseline where the baseline period is defined as six months prior to the index date, i.e., the date of the patient's first genomic test. Baseline covariates include age (in years), line of anti-EGFR therapy, smoking status (yes/no), gender (female/male), and the Van Walraven Elixhauser Comorbidity (ELIX) score specific to lung cancer patients (expressed as a weighted measure across multiple common comorbidities). As the cohort is based on real-world-data, it is not possible to directly align treatment start date with the patient's first genomic as is achievable in a prospective study. Therefore, days between the first genomic test and start of treatment was added as a covariate to serve as a statistical control and was set to zero days in the analysis to mimic a post treatment scenario. Patient mortality captured as alive vs deceased within the study timeframe was also included.

### Example 12—Exemplary Statistical Model

Described herein are mathematical details of a HCSREM which is malleable enough to capture variable nonlinear trends, and it allows for the direct incorporation of patient characteristics in the form of covariates. In addition to these properties, this model can provide a unique corresponding temporal ctDNA pattern for each combination of covariate values. It is the ability to provide this type of patient-specific information that makes this methodology attractive in targeted oncology efforts.

The model is partitioned into first- and second-level equations, which create the hierarchical structure. The first-level equation assumes the form of a truncated cubic spline and captures how a particular patient's ctDNA levels change over time (see equation (1)). At a high level this is achieved by creating a function that is split into various pieces that traverse the abscissa. Within each piece, a cubic polynomial is used to fit the data where the ends of the consecutive cubic polynomials are connected by knots. Knot location, as well as the number of knots can be strategically devised based on data inspection, though “automated” methods for determining knot quantity and placement exist. Ultimately, a cubic spline model combines the separate pieces to form a single uniform function to represent the data.

$${Y}_{ij} = {π}_{0 i} + {π}_{1 i} {t}_{ij} + {π}_{2 i} {t}_{ij}^{2} + {π}_{3 i} {t}_{ij}^{3} + {\sum }_{k = 1}^{K} {{π}_{( k + 3 ) i} ( {t}_{ij} - {ϵ}_{k} )}_{+}^{3} + {ε}_{ij} \qquad ( Equation 1 )$$

$$where {( t - ϵ )}_{+} = \{ \begin{matrix}0 & if t \leq ϵ \\ t - ϵ & if t > ϵ\end{matrix} \}$$

In equation (1), ctDNA measurements (or a transformation thereof) captured over time are represented by the

$${Y}_{ij}^{′} s ,$$

where i is used to index patients and j indexes the measurement occasion. Timepoints captured within the patient are given by tᵢⱼ, ∈ is the value of the k^{th} knot, the

$${π}_{ri}^{′} s$$

are the r response parameters, where each i.e. π₀ᵢ, π₁ᵢ, . . . , π₍ₖ₊₃₎ᵢ varies across patients i.e. the random effects, and, εᵢⱼ is the error term and is assumed to be normally distributed with a mean of 0 and variance σ². The response parameters are especially important as they collectively govern the shape of each patient's unique longitudinal ctDNA trajectory and serve to bridge the first and second-level equations.

The significance of the second-level equations is they contain information about individual patient characteristics and associate these characteristics with the response parameters themselves. The second-level equations are given below.

$${π}_{ri} = {β}_{r 0} + {\sum }_{c = 1}^{{C}_{r}} {β}_{rc} {X}_{ci} + {e}_{ri} \qquad ( Equation 2 )$$

where, X_{ci} represents a desired patient characteristic, β_{rc} captures the linear relationship between the response parameter and the patient characteristic, βᵣ₀ is the intercept for each corresponding πᵣᵢ, and eᵣᵢ represents a random component and is assumed to adhere to the following multivariate normal distribution:

$$[ \begin{matrix}{e}_{0 i} \\ {e}_{1 i} \\ ⋮ \\ {e}_{ri}\end{matrix} ] ∼ MVN ( [ \begin{matrix}0 \\ 0 \\ ⋮ \\ 0\end{matrix} ] , [ \begin{matrix}{σ}_{{e}_{0 i}}^{2} & {σ}_{{e}_{0 i} {e}_{1 i}} & … & {σ}_{{e}_{0 i} {e}_{ri}} \\ {σ}_{{e}_{0 i} {e}_{1 i}} & {σ}_{{e}_{1 i}}^{2} & … & {σ}_{{e}_{1 i} {e}_{ri}} \\ ⋮ & ⋮ & ⋱ & ⋮ \\ {σ}_{{e}_{0 i} {e}_{ri}} & {σ}_{{e}_{1 i} {e}_{ri}} & … & {σ}_{{e}_{ri}}^{2}\end{matrix} ] ) .$$

When the model contains covariates, it is referred to as a conditional model, otherwise it is an unconditional model. The unconditional model provides results at the cohort level and the conditional model is responsible for producing patient-level results.

Further, the described velocity plots, which are useful when examining the direction and speed in which ctDNA levels change at a given point in time i.e. the instantaneous rate of change (IRC) is of interest. Each model generated patient trajectory has at its heart, a cubic spline. An advantageous property of cubic splines is they are twice differentiable, thus, the IRC at a given time point can be calculated. 20 In the case of the adopted spline model, this amounts to taking the first derivative of equation (1) with respect to time resulting in:

$$\frac{{dY}_{ij}}{{dt}_{ij}} = {π}_{1 i} + 2 {π}_{2 i} {t}_{ij} + 3 {π}_{3 i} {t}_{ij}^{2} + {\sum }_{k = 1}^{K} 3 {{π}_{( k + 3 ) i} ( {t}_{ij} - {ϵ}_{k} )}_{+}^{2} \qquad ( Equation 3 )$$

The value of the IRC is given by the slope of the line tangent to the patient trajectory, where positive values correspond to an increasing IRC, negative values to decrease, and IRC values of zero indicate either a peak or trough was reached, or that the trajectory is flat. The further the IRC value is from zero, the more extreme the rate of change is.

### Example 13—Statistical Analysis and Results

Data were extracted using SAS software package 9.4 (SAS Institute, Cary, NC, USA) and all statistical analysis for the HCSREM was performed using R version 4.1.3. A total of 400 patients with advanced NSCLC were identified from the GuardantINFORM database as having at least three G360 tests. Seventy-three patients were excluded as their first test was more than 120 days after therapy initiation and five were excluded due to germline mutations. Of the remaining patients, 163 received anti-EGFR therapy with a total of 561 ctDNA longitudinal measurements, where these 163 patients defined the cohort used in the analysis. The average age of these patients was 62 years, 66% of them were females, average line of anti-EGFR therapy was 1 and the average time between G360 test and treatment initiation was 0 days (range −115 days to 30 days) (Table 1).

TABLE 4
Summary of Patient Characteristics

| | | %/Standard |
|---|---|---|
| Characteristics (Total N = 163) | N/Mean | Deviation |
| Age (years) | 61.18 | 10.88 |
| Female | 108 | 66% |
| ELIX score | 1.89 | 1.86 |
| Current or prior smoker | 123 | 75% |
| Line of anti-EGFR therapy | 1.44 | 0.99 |
| Time between G360 test and treatment | 0.29 | 31.98 |
| initiation (days) | | |
| ctDNA (%)* | 5.66 | 10.59 |
| Deceased at the end of study period | 55 | 33% |

*ctDNA value was extracted from each test and summarized, thus includes multiple ctDNA values for each patient

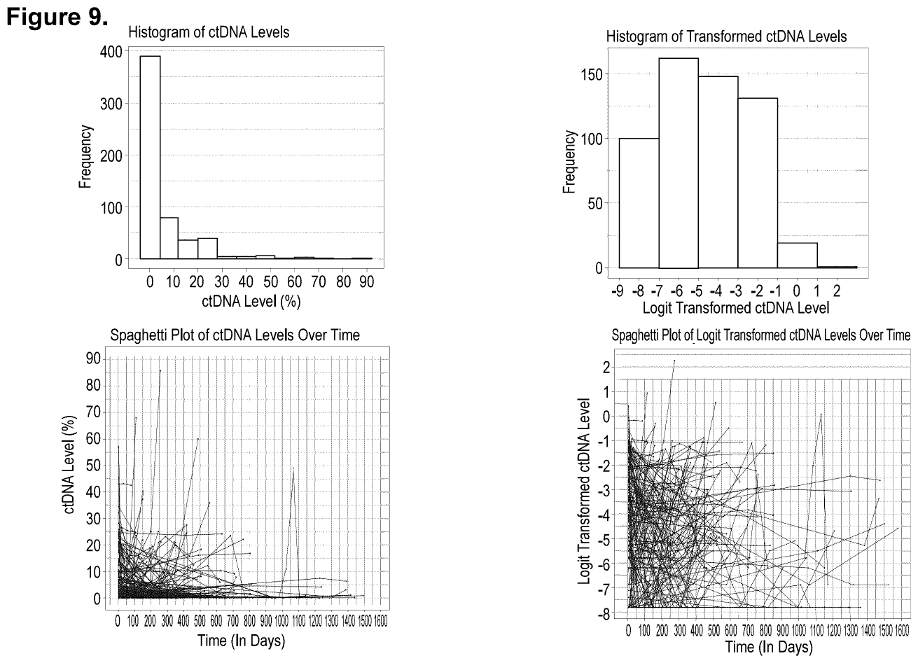

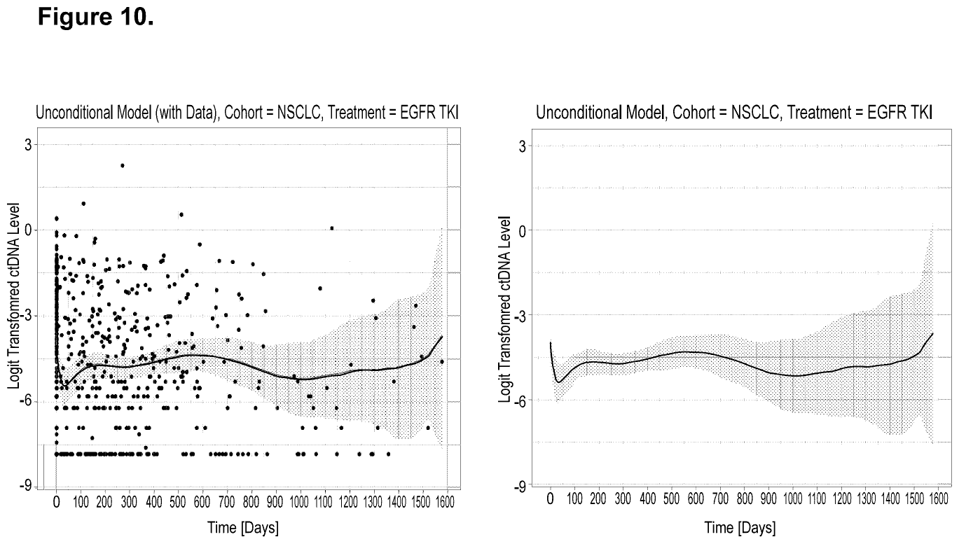

The Inventors developed an unconditional model fit to transformed data using knots set at 50, 125, 250, 500, 750, 1000, and 1250 days respectively as shown in FIG. 9. To ensure consistency, other knot orientations were explored, although different orientations did little to alter results. Results are presented graphically as spline model parameter estimates are difficult to interpret although parameter estimates and related output are provided in the supplemental information for reference. The graphical manifestation of the unconditional model, referred to as a response pattern, is presented in FIG. 10. Here, a black curve denotes the response pattern for the cohort, while each black dot indicates a ctDNA level value. The purple region represents the 95% confidence bands of the estimated trajectory.

The response pattern suggests ctDNA levels drop substantially between the first G360 test and 30 days, then rise rapidly until 150 days, at which point ctDNA levels dip slightly and rise again at around 300 days, although at a less extreme rate. Additionally, from 550 days to 1000 days ctDNA levels drop, and then rise again from 1000 to 1600 days. The corresponding 95% confidence band expand over time as the number of datapoints decreases. The flexibility built into the unconditional model revealed details hidden within the data that simpler models would not detect. Despite this, the unconditional model only estimates the response pattern for the cohort and does not account for the contingency that patients with different characteristics may exhibit different response patterns. To assess the impact of incorporating patient characteristics, a conditional model that incorporated all baseline covariates was fit to the data. As is typical in hierarchical models, all numerical covaries were centered about their respective means.

### Example 14—Age and Health Status, Response Patterns

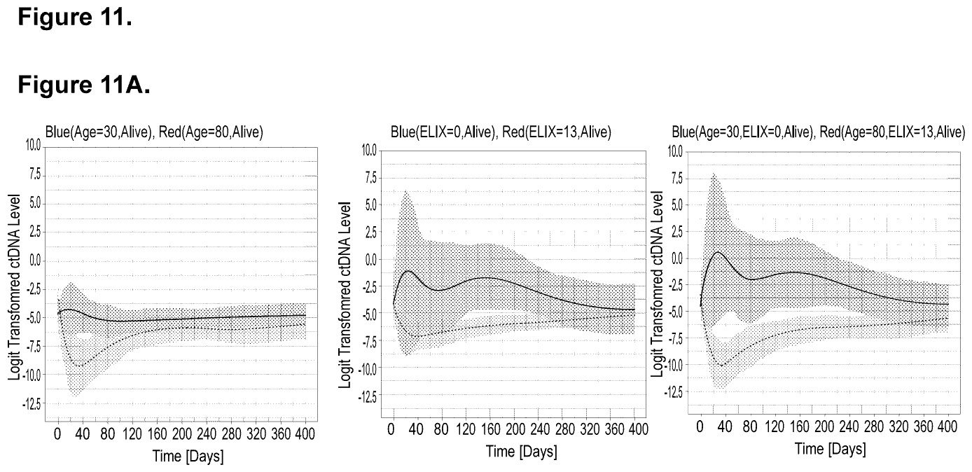

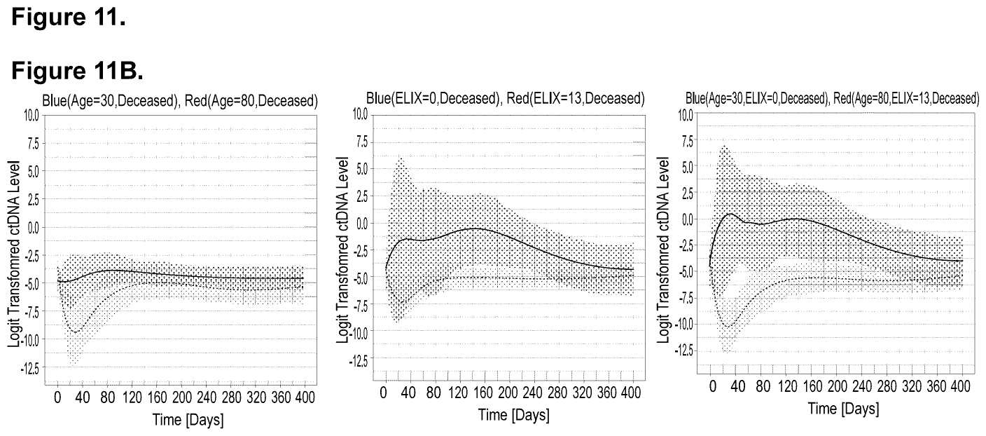

Here, FIG. 11 show how baseline age and health status, as measured by the ELIX score, impact response patterns in female non-smokers receiving their first line of EGFR-TKI treatment. Results are separated by patients who are alive vs deceased. As data becomes sparse after 400 days, we examine the first 400 days only. Examples presented above reveal that patients with different characteristics have different response patterns. In the top-left panel, the response curve for a 30 and 80-year-old with average ELIX scores are contrasted.

These results suggest 80-year-old patients did not exhibit initial post treatment drop in ctDNA levels in comparison with 30-year-old patients who demonstrated a rapid decrease followed by a rapid increase. The top-middle panel indicates response patterns for patients with an average age and a maximum ELIX score of 13 appear to be quite different compared to the same patient with a minimum ELIX score of 0, implying patients with many comorbidities exhibited a delayed treatment response. In the top-right panel, response patterns are displayed for older patients with high comorbidity burden and younger patients who are otherwise healthy, illustrating how the age/health status combination amplifies the disparity in response patterns. Though not shown, beyond 400 days, a decreasing trend in ctDNA values is observed for patients who remained alive at the end of the study while the trend increases for patients who died before study end.

### Example 15—Velocity Plots

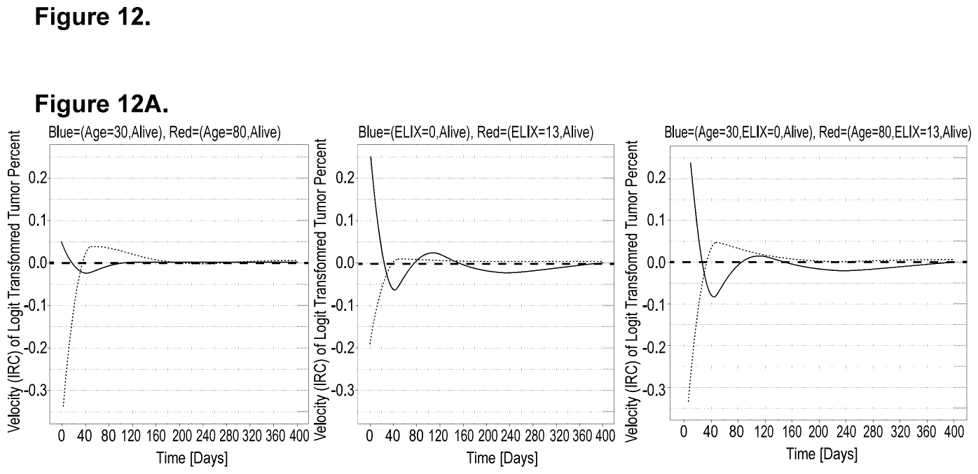

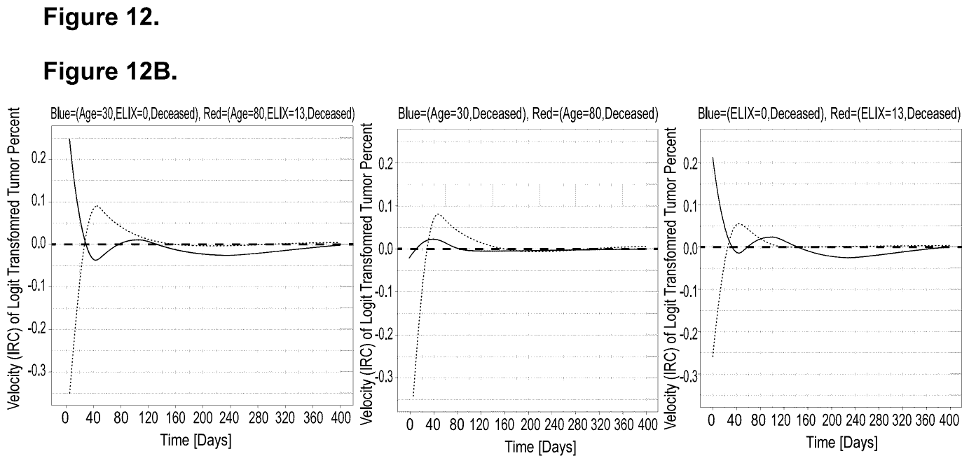

To focus on the response pattern's behavior, velocity plots that display the IRC for a corresponding response pattern were generated (FIG. 12). Information presented in a velocity plot can be gleaned from the response patterns themselves, but the differences in the response patterns are accentuated when examining them through an IRC lens. Thus, comparing velocity plots can provide additional clues as to where response patterns are similar and where they diverge based on the IRC value. Another advantage of utilizing velocity plots occurs when baseline values between response patterns are dissimilar and therefore differences between response patterns may be due to the fact that biomarker values were different at the onset. In these instances, using velocity plots to make comparisons may be more appropriate as the IRC is invariant to the biomarker's baseline value.

Interpreting a velocity plot is appreciated by one of ordinary skill in the art. Here, one can focus on the far-left panels. In the first 100 days, velocity plots for 80-year-old alive and deceased patients (red curves) exhibited different patterns. For survivors, the IRC was initially positive but slowed to zero around 20 days (indicating a peak in the corresponding response curve as referenced by the dashed line), and then decreased, where the fastest rate of decrease (−0.026 logits per day) occurred around 43 days. Beyond 43 days the IRC continued to decrease and remained relatively flat past 100 days. In contrast, the velocity plot for 80-year-old deceased patients displays a nearly opposite pattern.

### Example 16—Discussion

Described herein are methods and techniques that accommodate the analysis of complex longitudinal genomic data. As shown, the Inventors analyzed observational data and demonstrated use for application in different data settings, also including hypothesis generation, statistical inference, and patient monitoring. Here, the Inventors utilized 95% confidence bands fail to retain their traditional inferential meaning and instead are used as ‘guidelines’ in identifying differences in response patterns. This supported generation of thousands of response patterns.

One of ordinary skill readily appreciate that the described framework can be applied to representative cohorts as well. If statistical inference is the goal, since the potential exists to generate and compare numerous response patterns, the number of comparisons should be minimized, based on a priori hypotheses, and common considerations such as controlling for type-I error should be made. Hypotheses may include comparing response patterns between patients with pre-determined sets of covariate values (where other study covariates can be used as statistical controls) but can also include hypothesizing about the nature of the relationship between response pattern behavior and the covariate values themselves.

Another example includes patient monitoring. The general idea is each response pattern is a reasonable portrayal of a patient as described by his or her own unique set of characteristics—and—in this way—the same response pattern can serve as a reference for a new patient that shares these characteristics. Additionally, if survival status (deceased or not) is incorporated into the model, a reference response pattern for survivors and non-survivors can be created. Thus, if the response pattern of a new patient is consistent with that of a survivor, intervention is unnecessary, but if the response pattern mirrors that of a non-survivor, intervention may be required. Utilizing velocity plots to compare response patterns can further enhance this process-especially if baseline values between response patterns are dissimilar. To ensure reliable classification, such a monitoring system should undergo internal and external validation. Internal validation may be achieved by creating training and test datasets and then apply say k-fold cross-validation to assess classification accuracy. If an acceptable level of accuracy is achieved, external validation can be accomplished if new patients i.e. not involved in cross-validation are also classified with a high degree of accuracy.

As described, changes in ctDNA levels can fluctuate significantly over time from patient to patient. Here, the aforementioned methods and technique generate patient-level results, where such results reveal ctDNA dynamics for clinical decision-making.

1. A method of determining a patient response in at least one patient, comprising,
obtaining nucleic acid sequence information from at least one patient, comprising measurements of temporal changes in a biomarker; and
determining a patient response for the at least one patient.

2. The method of claim 1, wherein the biomarker comprises circulating tumor DNA (ctDNA).

3. The method of claim 1 of any preceding claim, wherein the biomarker comprises allele frequency and tumor fraction.

4. The method of claim 1 of any preceding claim, wherein determining a patient response for the at least one patient comprises use of a database.

5. The method of claim 4, wherein the database comprises medical records and/or insurance records.

6. The method of claim 5, wherein use of the database comprises application of a model.

7. The method of claim 6, wherein the model is a hierarchal model.

8. The method of claim 6, wherein the model is an effects model.

9. The method of claim 6, wherein the model is a regression model.

10. The method of claim 6, wherein the model is a joint model.

11. The method of claim 7, wherein the hierarchal model is a hierarchical random effects model.

12. The method of claim 6, wherein the model comprises a cubic spline.

13. The method of claim 6 m, wherein the model comprises a regression model.

14. The method of claim 11, wherein the hierarchal random effects model comprises generation of data from nucleic acid sequence information comprising temporal changes in a biomarker comprising circulating tumor DNA (ctDNA) from at least one subject in a plurality of subjects.

15. The method of claim 14, wherein the generation of data comprises generation of a cubic spline for at least one subject in a plurality of subjects.

16. The method of claim 14, wherein the generation of data comprises generation of response parameters comprising one or more covariates.

17. The method of claim 14, wherein the generation of data comprises generation of response parameters without covariates.

18. The method of claim 17, wherein the response parameters apply a multivariate normal distribution.

19. The method of claim 1, wherein the determining a patient response for the at least one patient comprises generation of a velocity plot.

20. The method of claim 1, wherein the determining a patient response for the at least one patient comprises comparison to the model.

21. The method of claim 10, wherein the joint model comprises at least two models.

22. The method of claim 10, wherein the joint model comprises association factors between the at least two models.

23. The method of claim 10, wherein the joint model comprises a cubic spline and a proportional hazard model.

24. The method of claim 1, wherein the biomarker is measured with next-generation DNA sequencing.

25. The method of claim 24, wherein next-generation DNA sequencing comprising ligation of non-unique barcodes to the ctDNA.

26. The method of claim of 24, wherein next-generation DNA sequencing comprising ligation of unique barcodes to the ctDNA.

27. The method of claim of 24, wherein next-generation DNA sequencing comprising ligation of non-unique barcodes to ctDNA fragments, wherein the non-unique barcodes are present in at least 20×, at least 30×, at least 50×, or at least 100× molar excess.

28. A method of determining a patient response in at least one patient, comprising,
obtaining nucleic acid sequence information from at least one patient, comprising measurements of temporal changes in a biomarker comprising circulating tumor DNA (ctDNA); and
determining a patient response for the at least one patient comprising use of a database comprising medical records and/or insurance record from a plurality of subjects wherein use of the database comprises application of a hierarchal random effects model.

29. The method of claim 28, wherein the hierarchal random effects model comprises generation of data from nucleic acid sequence information comprising temporal changes in ctDNA from at least one subject in a plurality of subjects.

30. The method of claim 28, wherein the hierarchal random effects model comprises generation of a cubic spline for at least one subject in the plurality of subjects.

31. The method of claim 28, wherein the hierarchal random effects model comprises response parameters comprising one or more covariates for at least one subject in the plurality of subjects.

32. The method of claim 28, wherein the database comprises medical records and/or insurance records for the plurality of subjects.

33. A method of determining a patient response in at least one patient, comprising,
obtaining nucleic acid sequence information from at least one patient, comprising
measurements of temporal changes in a biomarker comprising circulating tumor DNA (ctDNA); and
determining a patient response for the at least one patient comprising use of a database comprising medical records and/or insurance record from a plurality of subjects wherein use of the database comprises application of a joint model comprising a cubic spline and proportional hazard model generated from data from nucleic acid sequence information for at least one subject in a plurality of subjects.

34. The method of claim 33, wherein the database comprises medical records and/or insurance records for the plurality of subjects.

35. A system comprising a machine comprising at least one processor and storage comprising instructions capable of performing the method of claim 1.

36. A computer readable medium comprising instructions capable of performing the method of claim 1.
